SEQUENCE LISTING (23 sequences)

SEQ ID NO: 1 - protein, 242 aa, Artificial Sequence
  Synthesized
DVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIKGGGGSGGGGSGGGGSQVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAILGYFDYWGQGTMVTVSS

SEQ ID NO: 2 - protein, 241 aa, Artificial Sequence
  Synthesized
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 3 - protein, 242 aa, Artificial Sequence
  Synthesized
DVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIKGGGGSGGGGSGGGGSQVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAIIGYFDYWGQGTMVTVSS

SEQ ID NO: 4 - protein, 241 aa, Artificial Sequence
  Synthesized
DILLTQSPVILSVSPGERVSFSCRASQSIGTNIHWYQQRTDQSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSEDTAIYYCARALTYYDYEFAYWGQGTLVTVSA

SEQ ID NO: 5 - protein, 241 aa, Artificial Sequence
  Synthesized
DILLTQSPVILSVSPGERVSFSCRASQSIGTNIHWYQQKPDQSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLRAEDTAIYYCARALTYYDYEFAYWGQGTLVTVSA

SEQ ID NO: 6 - protein, 10 aa, Artificial Sequence
  Synthesized
GFSLTNYGVH

SEQ ID NO: 7 - protein, 16 aa, Artificial Sequence
  Synthesized
VIWSGGNTDYNTPFTS

SEQ ID NO: 8 - protein, 11 aa, Artificial Sequence
  Synthesized
ALTYYDYEFAY

SEQ ID NO: 9 - protein, 11 aa, Artificial Sequence
  Synthesized
RASQSIGTNIH

SEQ ID NO: 10 - protein, 11 aa, Artificial Sequence
  Synthesized
RASQSIGTNIH

SEQ ID NO: 11 - protein, 9 aa, Artificial Sequence
  Synthesized
QQNNNWPTT

SEQ ID NO: 12 - protein, 10 aa, Artificial Sequence
  Synthesized
GFTFTTYYIS

SEQ ID NO: 13 - protein, 17 aa, Artificial Sequence
  Synthesized
YINMGSGGTNYNEKFKG

SEQ ID NO: 14 - protein, 6 aa, Artificial Sequence
  Synthesized
LGYFDY

SEQ ID NO: 15 - protein, 16 aa, Artificial Sequence
  Synthesized
RSSQSLLDSDGGTYLY

SEQ ID NO: 16 - protein, 7 aa, Artificial Sequence
  Synthesized
LVSTLGS

SEQ ID NO: 17 - protein, 9 aa, Artificial Sequence
  Synthesized
MQLTHWPYT

SEQ ID NO: 18 - protein, 6 aa, Artificial Sequence
  Synthesized
IGYFDY

SEQ ID NO: 19 - protein, 568 aa, Artificial Sequence
  Synthesized
DVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIKGGGGSGGGGSGGGGSQVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAILGYFDYWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 20 - protein, 348 aa, Artificial Sequence
  Synthesized
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 568 aa, Artificial Sequence
  Synthesized
DVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIKGGGGSGGGGSGGGGSQVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAIIGYFDYWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 22 - protein, 348 aa, Artificial Sequence
  Synthesized
DILLTQSPVILSVSPGERVSFSCRASQSIGTNIHWYQQRTDQSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSEDTAIYYCARALTYYDYEFAYWGQGTLVTVSARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 348 aa, Artificial Sequence
  Synthesized
DILLTQSPVILSVSPGERVSFSCRASQSIGTNIHWYQQKPDQSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLRAEDTAIYYCARALTYYDYEFAYWGQGTLVTVSARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2020/0354460 A1
(43) Pub. Date: Nov. 12, 2020

(54) Bispecific antibodies against EGFR and PD-1

(71) Applicant: WUXI BIOLOGICS IRELAND LIMITED., Dublin 1 (IE)

(72) Inventors: Zhuozhi WANG, Shanghai (CN); Jing LI, Lexington, MA (US)

(21) Appl. No.: 16/652,000

(22) PCT Filed: Sep. 26, 2018
(86) PCT No.: PCT/CN2018/107582

§ 371 Date: Mar. 27, 2020

(30) Foreign Application Priority Data
Sep. 29, 2017 (CN) PCT/CN2017/104584

(51) Int. Cl. C07K 16/28; A61P 35/00

(52) U.S. Cl. CPC C07K 16/2863; C07K 16/2818; A61K 2039/505; C07K 2317/31; A61P 35/00

(57) ABSTRACT

The present invention provides bispecific antibodies comprising first binding domain which binds to EGFR and a second binding domain which binds to PD-1, wherein the antibody or the antigen binding-fragment is in a format selected from the group consisting of single chain Fv (scFv), diabodies, and oligomers of the foregoing formats. The present invention further provides amino acid sequences of the antibodies of the invention, cloning or expression vectors, host cells and methods for expressing or isolating the antibodies. Therapeutic compositions comprising the antibodies of the invention are also provided. The invention also provides methods for treating cancers and other diseases with the bispecific antibodies.

### CROSS REFERENCE TO RELATED APPLICATION

This application is a U.S. National Stage entry of PCT Application No: PCT/CN2018/107582 filed on Sep. 26, 2018 which claims the benefit of and priority to PCT patent application serial number PCT/CN2017/104584, filed Sep. 29, 2017, the contents of which are hereby incorporated by reference.

### TECHNICAL FIELD

The present invention relates to bispecific antibodies comprising a first binding domain which binds to EGFR and a second binding domain which binds to PD-1, wherein the antibody or the antigen binding-fragment is in a format selected from the group consisting of single chain Fv (scFv), diabodies, and oligomers of the foregoing formats. Moreover, the invention provides a polynucleotide encoding the antibodies, a vector comprising said polynucleotide, a host cell, a process for the production of the antibodies and immunotherapy in the treatment of cancer, infections or other human diseases using the bispecific antibodies.

### BACKGROUND OF THE INVENTION

Epidermal growth factor receptor (EGFR) is overexpressed in a variety of human cancers. EGFR can be activated by different ligands. Among these ligands, EGF is high affinity ligands of EGFR. EGF-binding to extracellular domain of EGFR induces the dimerization of the receptor. EGFR may also pair with another member of ErbB receptors, such as Her2, forming heterodimer. EGFR dimerization stimulates its intrinsic kinase activity and subsequent phosphorylation of EGFR at several sites. This phosphorylation elicits downstream activation and signaling, and further initiates several signal transduction cascades, principally MAPK, Akt and JNK pathways, leading to DNA synthesis and cell proliferation. Overall EGF/EGFR pathway induces cell differentiation, migration, adhesion and proliferation. Due to overexpression of EGFR in a variety of human cancers, EGFR represents an important target for targeted therapy.

Two EGFR-targeted antibodies, cetuximab (Erbitux) and panitumumab (Vectibix), have been approved by the US Food and Drug Administration for the treatment of colon cancers and head and neck cancers. These antibodies block the binding of ligands to EGFR and downstream signals, and mediate antitumor immune responses.

Programmed Death-1 (PD-1, CD279) is a member of CD28 family expressed on activated T cells and other immune cells. Engagement of PD-1 inhibits function in these immune cells. PD-1 has two known ligands, PD-L1 (B7-H1, CD274) and PD-L2 (B7-DC, CD273), both belong to B7 family. PD-L1 expression is inducible on a variety of cell types in lymphoid and peripheral tissues, whereas PD-L2 is more restricted to myeloid cells including dendritic cells. The major role of PD-1 pathway is to tune down inflammatory immune response in tissues and organs.

It is found that cancer cells are capable of evading immune destruction by upregulating PD-1/PD-L1 pathway in the tumor microenvironment [Boussiotis 2016 N Engl J Med]. This mechanism is in particular found in tumors with activating mutations in the EGFR gene. It is possible that PD-1 pathway upregulation is a typical mechanism of immune evasion. As an evidence, high PD-L1 expression is found in tumors of patients with EGFR mutations [Azuma 2014 Ann Oncol; Ramalingam 2016 J Thorac Oncol].

In fact, anti-EGFR antibodies haven't been approved for lung cancer therapy although EGFR overexpression has been found in lung cancers. Initial effectiveness of anti-EGFR therapy frequently has been dampened by resistance to such targeted therapy, mainly due to EGFR mutations. It is unknown that targeting both EGFR pathway and PD-1/PD-L1 pathway may provide more effective therapy than targeting EGFR alone for treatment of various tumors. Thus, the goal of this project is to generate bispecific antibodies against both EGFR and PD-1 and prove that the antibodies provide several benefits in cancer therapy. First the bispecific antibody may be used for lung cancer therapy, whereas anti-EGFR antibodies haven't been approved for this indication which EGFR overexpression has been found. Second, the bispecific antibody may reverse the resistance of anti-EGFR therapy. Also compared with anti-PD-1 therapy, the bispecific antibody may increase the response rate on PD-L1 and EGFR double positive cancers.

### SUMMARY OF THE INVENTION

The present invention provides isolated antibodies, in particular bispecific antibodies.

In one aspect, the present invention provides a bispecific antibody or an antigen binding fragment thereof, comprising a first binding domain which binds to human EGFR and a second binding domain which binds to human PD-1, wherein the antibody or the antigen binding-fragment comprises a format selected from the group consisting of single chain Fv (scFv), diabodies, and oligomers of the foregoing formats.

In one embodiment, the antibody or the antigen binding-fragment is in a format selected from the group consisting of single chain Fv (scFv), diabodies, and oligomers of the foregoing formats.

The aforesaid antibody or the antigen binding-fragment, wherein the second binding domain binds to murine PD-1.

In one embodiment, the present invention provides an antibody or an antigen binding fragment thereof, wherein the antibody comprises single chain Fv against EGFR.

In one embodiment, the present invention provides an antibody or an antigen binding fragment thereof, wherein the antibody comprises single chain Fv against PD-1.

In one embodiment, the present invention provides an antibody or an antigen binding fragment thereof, wherein the antibody comprises single chain Fv against EGFR and single chain Fv against PD-1.

The aforesaid antibody or an antigen binding fragment thereof, wherein the antibody or the antigen binding-fragment

a) binds to human EGFR with a K_{D} of 5.45E-10 or less; and

b) binds to human PD-1 with a K_{D} of 1.98E-09 or less.

The aforesaid antibody or an antigen binding fragment thereof, exhibits at least one of the following properties:

a) binds to human EGFR with a K_{D} of between 5.45E-10 and 5.49E-10; and

b) binds to human PD-1 with a K_{D} of between 1.98E-09 and 7.68E-09.

The aforesaid antibody or an antigen binding fragment thereof, comprising:

a polypeptide chain comprising the first binding domain, the first binding domain comprises a VH region and a VL region against EGFR;

another polypeptide chain comprising the second binding domain, the second binding domain comprises a VH region and a VL region against PD-1.

In one embodiment, the aforesaid antibody or an antigen binding fragment thereof, wherein the first binding domain comprises

a VH region comprising H-CDR1, H-CDR2, H-CDR3 and a VL region comprising L-CDR1, L-CDR2, L-CDR3; wherein

the H-CDR3 comprises a sequence as depicted in SEQ ID NO: 8, and conservative modifications thereof, the H-CDR2 comprises a sequence as depicted in SEQ ID NO: 7, and conservative modifications thereof; the H-CDR1 comprises a sequence as depicted in SEQ ID NO: 6, and conservative modifications thereof, and

the L-CDR3 comprises a sequence as depicted in SEQ ID NO: 11, and conservative modifications thereof, the L-CDR2 comprises a sequence as depicted in SEQ ID NO: 10, and conservative modifications thereof; the L-CDR1 comprises a sequence as depicted in SEQ ID NO: 9, and conservative modifications thereof.

The aforesaid antibody or an antigen binding fragment thereof, comprising an amino acid sequence that is at least 70%, 80%, 90%, 95% or 99% homologous to a sequence selected from a group consisting of SEQ ID NOs: 1-5.

The aforesaid antibody or an antigen binding fragment thereof, comprising an amino acid sequence selected from a group consisting of SEQ ID NOs: 1-5.

The aforesaid antibody or an antigen binding fragment thereof, comprising:

a) a variable region of the second binding domain having an amino acid sequence that is at least 70%, 80%, 90%, 95% or 99% homologous to a sequence selected from a group consisting of SEQ ID NOs: 1, 3; and

b) a variable region of the first binding domain having an amino acid sequence that is at least 70%, 80%, 90%, 95% or 99% homologous to a sequence selected from a group consisting of SEQ ID NOs: 2, 4, 5.

The aforesaid antibody or an antigen binding fragment thereof, comprising:

a) a variable region of the second binding domain having an amino acid sequence selected from the group consisting of SEQ ID NOs: 1, 3; and

b) a variable region of the first binding domain having an amino acid sequence selected from the group consisting of SEQ ID NOs:2, 4, 5.

In various embodiments, the aforesaid antibody or an antigen binding fragment thereof comprises:

a) a variable region of the second binding domain having an amino acid sequence selected from the group consisting of SEQ ID NO: 1; and

b) a variable region of the first binding domain having an amino acid sequence selected from the group consisting of SEQ ID NO: 2;

or the aforesaid antibody or an antigen binding fragment thereof comprises:

a) a variable region of the second binding domain having an amino acid sequence selected from the group consisting of SEQ ID NO: 3; and

b) a variable region of the first binding domain having an amino acid sequence selected from the group consisting of SEQ ID NO: 2;

or the antibody or an antigen binding fragment thereof comprises:

a) a variable region of the second binding domain having an amino acid sequence selected from the group consisting of SEQ ID NO: 1; and

b) a variable region of the first binding domain having an amino acid sequence selected from the group consisting of SEQ ID NO: 4;

or the antibody or an antigen binding fragment thereof comprises:

a) a variable region of the second binding domain having an amino acid sequence selected from the group consisting of SEQ ID NO: 1; and

b) a variable region of the first binding domain having an amino acid sequence selected from the group consisting of SEQ ID NO: 5;

or the antibody or an antigen binding fragment thereof comprises:

a) a variable region of the second binding domain having an amino acid sequence selected from the group consisting of SEQ ID NO: 3; and

b) a variable region of the first binding domain having an amino acid sequence selected from the group consisting of SEQ ID NO: 4;

or the antibody or an antigen binding fragment thereof comprises:

a) a variable region of the second binding domain having an amino acid sequence selected from the group consisting of SEQ ID NO: 3; and

b) a variable region of the first binding domain having an amino acid sequence selected from the group consisting of SEQ ID NO: 5.

The sequence of said antibody is shown in Table 1 and Sequence Listing.

TABLE 1
Deduced amino acid sequences of the antibodies

| | | SEQ | |
|---|---|---|---|
| Clone ID | | ID NO | Amino acid sequence |
| WBP336B = | variable region | 1 | DVVMTQSPLSLPVTLGQPASISCRSSQSLLDSDGGTY |
| W336- | (underlined VL | | LYWFQQRPGQSPRRLIYLVSTLGSGVPDRFSGSGSGT |
| T1U2.G10- | and VH) of | | DFTLKISRVEAEDVGVYYCMQLTHWPYTFGQGTKLEI |
| 4.uhIgG4.SP | anti-PD-1 | | KGGGGSGGGGSGGGGSQVQLVQSGAEVKKPGSSVKVS |
| (dK) | binding domain | | CKASGFTFTTYYISWVRQAPGQGLEYLGYINMGSGGT |
| | | | NYNEKFKGRVTITADKSTSTAYMELSSLRSEDTAVYY |
| | | | CAILGYFDYWGQGTMVTVSS |
| | variable region | 2 | DILLTQSPVILSVSPGERVSFSCRASQSIGTNIHWYQ |
| | (underlined VL | | QRTNGSPRLLIKYASESISGIPSRFSGSGSGTDFTLS |
| | and VH) of | | INSVESEDIADYYCQQNNNWPTTFGAGTKLELKGGGG |
| | anti-EGFR | | SGGGGSGGGGSQVQLKQSGPGLVQPSQSLSITCTVSG |
| | binding domain | | FSLTNYGVHWVRQSPGKGLEWLGVIWSGGNTDYNTPF |
| | | | TSRLSINKDNSKSQVFFKMNSLQSNDTAIYYCARALT |
| | | | YYDYEFAYWGQGTLVTVSA |
| WBP336C = | variable region | 3 | DVVMTQSPLSLPVTLGQPASISCRSSQSLLDSDGGTY |
| W336- | (underlined VL | | LYWFQQRPGQSPRRLIYLVSTLGSGVPDRFSGSGSGT |
| T1U3.G10- | and VH) of | | DFTLKISRVEAEDVGVYYCMQLTHWPYTFGQGTKLEI |
| 4.uhIgG4.SP | anti-PD-1 | | KGGGGSGGGGSGGGGSQVQLVQSGAEVKKPGSSVKVS |
| (dK) | binding domain | | CKASGFTFTTYYISWVRQAPGQGLEYLGYINMGSGGT |
| | | | NYNEKFKGRVTITADKSTSTAYMELSSLRSEDTAVYY |
| | | | CAIIGYFDYWGQGTMVTVSS |
| | variable region | 2 | DILLTQSPVILSVSPGERVSFSCRASQSIGTNIHWYQ |
| | (underlined VL | | QRTNGSPRLLIKYASESISGIPSRFSGSGSGTDFTLS |
| | and VH) of | | INSVESEDIADYYCQQNNNWPTTFGAGTKLELKGGGG |
| | anti-EGFR | | SGGGGSGGGGSQVQLKQSGPGLVQPSQSLSITCTVSG |
| | binding domain | | FSLTNYGVHWVRQSPGKGLEWLGVIWSGGNTDYNTPF |
| | | | TSRLSINKDNSKSQVFFKMNSLQSNDTAIYYCARALT |
| | | | YYDYEFAYWGQGTLVTVSA |
| WBP336D | variable region | 1 | DVVMTQSPLSLPVTLGQPASISCRSSQSLLDSDGGTY |
| | (underlined VL | | LYWFQQRPGQSPRRLIYLVSTLGSGVPDRFSGSGSGT |
| | and VH) of | | DFTLKISRVEAEDVGVYYCMQLTHWPYTFGQGTKLEI |
| | anti-PD-1 | | KGGGGSGGGGSGGGGSQVQLVQSGAEVKKPGSSVKVS |
| | binding domain | | CKASGFTFTTYYISWVRQAPGQGLEYLGYINMGSGGT |
| | | | NYNEKFKGRVTITADKSTSTAYMELSSLRSEDTAVYY |
| | | | CAILGYFDYWGQGTMVTVSS |
| | variable region | 4 | DILLTQSPVILSVSPGERVSFSCRASQSIGTNIHWYQ |
| | (underlined VL | | QRTDQSPRLLIKYASESISGIPSRFSGSGSGTDFTLS |
| | and VH) of | | INSVESEDIADYYCQQNNNWPTTFGAGTKLELKGGGG |
| | anti-EGFR | | SGGGGSGGGGSQVQLKQSGPGLVQPSQSLSITCTVSG |
| | binding domain | | FSLTNYGVHWVRQSPGKGLEWLGVIWSGGNTDYNTPF |
| | | | TSRLSINKDNSKSQVFFKMNSLQSEDTAIYYCARALT |
| | | | YYDYEFAYWGQGTLVTVSA |
| WBP336E | variable region | 1 | DVVMTQSPLSLPVTLGQPASISCRSSQSLLDSDGGTY |
| | (underlined VL | | LYWFQQRPGQSPRRLIYLVSTLGSGVPDRFSGSGSGT |
| | and VH) of | | DFTLKISRVEAEDVGVYYCMQLTHWPYTFGQGTKLEI |
| | anti-PD-1 | | KGGGGSGGGGSGGGGSQVQLVQSGAEVKKPGSSVKVS |
| | binding domain | | CKASGFTFTTYYISWVRQAPGQGLEYLGYINMGSGGT |
| | | | NYNEKFKGRVTITADKSTSTAYMELSSLRSEDTAVYY |
| | | | CAILGYFDYWGQGTMVTVSS |
| | variable region | 5 | DILLTQSPVILSVSPGERVSFSCRASQSIGTNIHWYQ |
| | (underlined VL | | QKPDQSPRLLIKYASESISGIPSRFSGSGSGTDFTLS |
| | and VH) of | | INSVESEDIADYYCQQNNNWPTTFGAGTKLELKGGGG |
| | anti-EGFR | | SGGGGSGGGGSQVQLKQSGPGLVQPSQSLSITCTVSG |
| | binding domain | | FSLTNYGVHWVRQSPGKGLEWLGVIWSGGNTDYNTPF |
| | | | TSRLSINKDNSKSQVFFKMNSLRAEDTAIYYCARALT |
| | | | YYDYEFAYWGQGTLVTVSA |
| WBP336F | variable region | 3 | DVVMTQSPLSLPVTLGQPASISCRSSQSLLDSDGGTY |
| | (underlined VL | | LYWFQQRPGQSPRRLIYLVSTLGSGVPDRFSGSGSGT |
| | and VH) of | | DFTLKISRVEAEDVGVYYCMQLTHWPYTFGQGTKLEI |
| | anti-PD-1 | | KGGGGSGGGGSGGGGSQVQLVQSGAEVKKPGSSVKVS |
| | binding domain | | CKASGFTFTTYYISWVRQAPGQGLEYLGYINMGSGGT |
| | | | NYNEKFKGRVTITADKSTSTAYMELSSLRSEDTAVYY |
| | | | CAIIGYFDYWGQGTMVTVSS |
| | variable region | 4 | DILLTQSPVILSVSPGERVSFSCRASQSIGTNIHWYQ |
| | (underlined VL | | QRTDQSPRLLIKYASESISGIPSRFSGSGSGTDFTLS |
| | and VH) of | | INSVESEDIADYYCQQNNNWPTTFGAGTKLELKGGGG |
| | anti-EGFR | | SGGGGSGGGGSQVQLKQSGPGLVQPSQSLSITCTVSG |
| | binding domain | | FSLTNYGVHWVRQSPGKGLEWLGVIWSGGNTDYNTPF |
| | | | TSRLSINKDNSKSQVFFKMNSLQSEDTAIYYCARALT |
| | | | YYDYEFAYWGQGTLVTVSA |
| WBP336G | variable region | 3 | DVVMTQSPLSLPVTLGQPASISCRSSQSLLDSDGGTY |
| | (underlined VL | | LYWFQQRPGQSPRRLIYLVSTLGSGVPDRFSGSGSGT |
| | and VH) of | | DFTLKISRVEAEDVGVYYCMQLTHWPYTFGQGTKLEI |
| | anti-PD-1 | | KGGGGSGGGGSGGGGSQVQLVQSGAEVKKPGSSVKVS |
| | binding domain | | CKASGFTFTTYYISWVRQAPGQGLEYLGYINMGSGGT |
| | | | NYNEKFKGRVTITADKSTSTAYMELSSLRSEDTAVYY |
| | | | CAIIGYFDYWGQGTMVTVSS |
| | variable region | 5 | DILLTQSPVILSVSPGERVSFSCRASQSIGTNIHWYQ |
| | (underlined VL | | QKPDQSPRLLIKYASESISGIPSRFSGSGSGTDFTLS |
| | and VH) of | | INSVESEDIADYYCQQNNNWPTTFGAGTKLELKGGGG |
| | anti-EGFR | | SGGGGSGGGGSQVQLKQSGPGLVQPSQSLSITCTVSG |
| | binding domain | | FSLTNYGVHWVRQSPGKGLEWLGVIWSGGNTDYNTPF |
| | | | TSRLSINKDNSKSQVFFKMNSLRAEDTAIYYCARALT |
| | | | YYDYEFAYWGQGTLVTVSA |

The aforesaid antibody or an antigen binding fragment thereof, comprising an amino acid sequence that is at least 70%, 80%, 90%, 95% or 99% homologous to a sequence selected from a group consisting of SEQ ID NOs: 19-23.

The aforesaid antibody or an antigen binding fragment thereof, comprising an amino acid sequence selected from a group consisting of SEQ ID NOs: 19-23.

The aforesaid antibody or an antigen binding fragment thereof, comprising:

a) the second binding domain having an amino acid sequence that is at least 70%, 80%, 90%, 95% or 99% homologous to a sequence selected from a group consisting of SEQ ID NOs: 19, 21; and

b) the first binding domain having an amino acid sequence that is at least 70%, 80%, 90%, 95% or 99% homologous to a sequence selected from a group consisting of SEQ ID NOs: 20, 22, 23.

The aforesaid antibody or an antigen binding fragment thereof, comprising:

a) the second binding domain having an amino acid sequence selected from the group consisting of SEQ ID NOs: 19, 21; and

b) the first binding domain having an amino acid sequence selected from the group consisting of SEQ ID NOs: 20, 22, 23.

In various embodiments, the aforesaid antibody or an antigen binding fragment thereof comprises:

a) the second binding domain having an amino acid sequence selected from the group consisting of SEQ ID NO: 19; and

b) the first binding domain having an amino acid sequence selected from the group consisting of SEQ ID NO: 20;

or the aforesaid antibody or an antigen binding fragment thereof comprises:

a) the second binding domain having an amino acid sequence selected from the group consisting of SEQ ID NO: 21; and

b) the first binding domain having an amino acid sequence selected from the group consisting of SEQ ID NO: 20;

or the antibody or an antigen binding fragment thereof comprises:

a) the second binding domain having an amino acid sequence selected from the group consisting of SEQ ID NO: 19; and

b) the first binding domain having an amino acid sequence selected from the group consisting of SEQ ID NO: 22;

or the antibody or an antigen binding fragment thereof comprises:

a) the second binding domain having an amino acid sequence selected from the group consisting of SEQ ID NO: 19; and

b) the first binding domain having an amino acid sequence selected from the group consisting of SEQ ID NO: 23;

or the antibody or an antigen binding fragment thereof comprises:

a) the second binding domain having an amino acid sequence selected from the group consisting of SEQ ID NO: 21; and

b) the first binding domain having an amino acid sequence selected from the group consisting of SEQ ID NO: 22;

or the antibody or an antigen binding fragment thereof comprises:

a) the second binding domain having an amino acid sequence selected from the group consisting of SEQ ID NO: 21; and

b) the first binding domain having an amino acid sequence selected from the group consisting of SEQ ID NO: 23.

The sequence of said antibody is shown in Table 3 and Sequence Listing.

The aforesaid antibody or an antigen binding fragment thereof, comprising a complementarity-determining region (CDR) having an amino acid sequence selected from the group consisting of SEQ ID NOs: 6-18.

The aforesaid antibody, or an antigen binding fragment thereof, wherein the second binding domain comprises:

a VH region comprising H-CDR1, H-CDR2, H-CDR3 and a VL region comprising L-CDR1, L-CDR2, L-CDR3;

wherein the H-CDR3 comprises an amino acid sequence as depicted in SEQ ID NO: 14 or SEQ ID NO: 18, and conservative modifications thereof.

Preferably, wherein the L-CDR3 against PD-1 comprises an amino acid sequence as depicted in SEQ ID NO: 17, and conservative modifications thereof.

Preferably, wherein the H-CDR2 against PD-1 comprises an amino acid sequence as depicted in SEQ ID NO: 13, and conservative modifications thereof.

Preferably, wherein the L-CDR2 against PD-1 comprises an amino acid sequence as depicted in SEQ ID NO: 16, and conservative modifications thereof.

Preferably, wherein the H-CDR1 against PD-1 comprises an amino acid sequence as depicted in SEQ ID NO: 12, and conservative modifications thereof.

Preferably, wherein the L-CDR1 against PD-1 comprises an amino acid sequence as depicted in SEQ ID NO: 15, and conservative modifications thereof.

In more preferred embodiment, the aforesaid antibody or an antigen binding fragment thereof, wherein the second binding domain comprises:

a VH region comprising H-CDR1, H-CDR2, H-CDR3 and a VL region comprising L-CDR1, L-CDR2, L-CDR3; wherein

a) the H-CDR3 comprises an amino acid sequence as depicted in SEQ ID NO: 14 or SEQ ID NO: 18, and conservative modifications thereof,

b) the L-CDR3 against PD-1 comprises an amino acid sequence as depicted in SEQ ID NO: 17, and conservative modifications thereof;

c) the H-CDR2 against PD-1 comprises an amino acid sequence as depicted in SEQ ID NO: 13, and conservative modifications thereof;

d) the L-CDR2 against PD-1 comprises an amino acid sequence as depicted in SEQ ID NO: 16, and conservative modifications thereof;

e) the H-CDR1 against PD-1 comprises an amino acid sequence as depicted in SEQ ID NO: 12, and conservative modifications thereof;

f) the L-CDR1 against PD-1 comprises an amino acid sequence as depicted in SEQ ID NO: 15, and conservative modifications thereof.

A preferred antibody or an antigen binding fragment thereof, wherein the second binding domain comprises:

a) a H-CDR1 comprising SEQ ID NO: 12;

b) a H-CDR2 comprising SEQ ID NO: 13;

c) a H-CDR3 comprising SEQ ID NO: 14;

d) a L-CDR1 comprising SEQ ID NO: 15;

e) a L-CDR2 comprising SEQ ID NO: 16;

f) a L-CDR3 comprising SEQ ID NO: 17.

A preferred antibody or an antigen binding fragment thereof, wherein the second binding domain comprises:

a) a H-CDR1 comprising SEQ ID NO: 12;

b) a H-CDR2 comprising SEQ ID NO: 13;

c) a H-CDR3 comprising SEQ ID NO: 18;

d) a L-CDR1 comprising SEQ ID NO: 15;

e) a L-CDR2 comprising SEQ ID NO: 16;

f) a L-CDR3 comprising SEQ ID NO: 17.

The CDR sequences of said antibodies are shown in Table 2 and Sequence Listing.

TABLE 2
The CDR sequences of the antibodies

| | | SEQ ID | |
|---|---|---|---|
| Clone ID. | | NO | Amino acid sequence |
| WBP336B = | Anti-PD-1: | 12 | GFTFTTYYIS |
| W336- | HCDR1 | | |
| T1U2.G10- | Anti-PD-1: | 13 | YINMGSGGTNYNEKFKG |
| 4.uhIgG4.SP | HCDR2 | | |
| (dK) | Anti-PD-1: | 14 | LGYFDY |
| | HCDR3 | | |
| | Anti-PD-1: | 15 | RSSQSLLDSDGGTYLY |
| | LCDR1 | | |
| | Anti-PD-1: | 16 | LVSTLGS |
| | LCDR2 | | |
| | Anti-PD-1: | 17 | MQLTHWPYT |
| | LCDR3 | | |
| WBP336C = | Anti-PD-1: | 12 | GFTFTTYYIS |
| W336- | HCDR1 | | |
| T1U3.G10- | Anti-PD-1: | 13 | YINMGSGGTNYNEKFKG |
| 4.uhIgG4.SP | HCDR2 | | |
| (dK) | Anti-PD-1: | 18 | IGYFDY |
| | HCDR3 | | |
| | Anti-PD-1: | 15 | RSSQSLLDSDGGTYLY |
| | LCDR1 | | |
| | Anti-PD-1: | 16 | LVSTLGS |
| | LCDR2 | | |
| | Anti-PD-1: | 17 | MQLTHWPYT |
| | LCDR3 | | |

In more preferred embodiment, the aforesaid antibody, or an antigen binding fragment thereof, wherein the first binding domain comprises:

a) a H-CDR1 comprising SEQ ID NO: 6;

b) a H-CDR2 comprising SEQ ID NO: 7;

c) a H-CDR3 comprising SEQ ID NO: 8;

d) a L-CDR1 comprising SEQ ID NO: 9;

e) a L-CDR2 comprising SEQ ID NO: 10;

f) a L-CDR3 comprising SEQ ID NO: 11.

The antibody of the invention can be a chimeric antibody.

The antibody of the invention can be a humanized antibody, or a fully human antibody.

The antibody of the invention can be a rodent antibody.

In a further aspect, the invention provides a nucleic acid molecule encoding the antibody, or antigen binding fragment thereof.

The invention provides a cloning or expression vector comprising the nucleic acid molecule encoding the antibody, or antigen binding fragment thereof.

The invention also provides a host cell comprising one or more cloning or expression vectors.

In yet another aspect, the invention provides a process, comprising culturing the host cell of the invention and isolating the antibody.

In a further aspect, the invention provides pharmaceutical composition comprising the antibody, or the antigen binding fragment of said antibody in the invention, and one or more of a pharmaceutically acceptable excipient, a diluent or a carrier.

The invention provides an immunoconjugate comprising said antibody, or antigen-binding fragment thereof in this invention, linked to a therapeutic agent.

Wherein, the invention provides a pharmaceutical composition comprising said immunoconjugate and one or more of a pharmaceutically acceptable excipient, a diluent or a carrier.

The invention also provides a method of modulating an immune response in a subject comprising administering to the subject the antibody, or antigen binding fragment of any one of said antibodies in this invention.

The invention also provides the use of said antibody or the antigen binding fragment thereof in the manufacture of a medicament for the treatment or prophylaxis of an immune disorder or cancer.

The invention also provides a method of inhibiting growth of tumor cells in a subject, comprising administering to the subject a therapeutically effective amount of said antibody, or said antigen-binding fragment to inhibit growth of the tumor cells.

Wherein, the invention provides the method, wherein the tumor cells are of a cancer selected from a group consisting of melanoma, renal cancer, prostate cancer, breast cancer, colon cancer, lung cancer, bone cancer, pancreatic cancer, skin cancer, cancer of the head or neck, cutaneous or intraocular malignant melanoma, uterine cancer, ovarian cancer, and rectal cancer.

The Features and Advantages of this Invention

A bispecific antibody against both EGFR and PD-1 pathways may provide several benefits in cancer therapy. First the bispecific antibody may be used for lung cancer therapy, whereas anti-EGFR antibodies haven't been approved for this indication although EGFR overexpression has been found in lung cancers. Second, the bispecific antibody may reverse the resistance of anti-EGFR therapy. Also compared with anti-PD-1 therapy, the bispecific antibody may increase the response rate on PD-L1 and EGFR double positive cancers.

### BRIEF DESCRIPTION OF THE DRAWINGS

FIG. 1 shows schematic formats of tested bispecific antibodies.

FIG. 2 is a diagram showing the possible mechanisms of targeting EGFR and PD-1.

FIG. 3 shows SEC of purified WBP336B (a) and WBP336C (b).

FIG. 4 shows human PD-1-binding ELISA (a) and FACS (b).

FIG. 5 shows human EGFR-binding ELISA (a) and FACS (b).

FIG. 6 shows human EGFR- and PD-1-dual binding ELISA (a) and FACS (b, c, d).

FIG. 7 shows cynomolgus PD-1-binding ELISA.

FIG. 8 shows mouse PD-1-binding FACS.

FIG. 9 shows cynomolgus monkey EGFR-binding FACS.

FIG. 10 shows that the bispecific antibodies blocked human or mouse PD-1 binding to PDL1 using ELISA (a) and FACS (b, c).

FIG. 11 shows that the bispecific antibodies blocked human EGF binding to EGFR in FACS.

FIG. 12 shows IL2 and IFNgamma release in human MLR assay.

FIG. 13 shows that the bispecific antibodies inhibited EGFR phosphorylation in A431 cells.

FIG. 14 shows the ADCC effect on EGFR+ tumor cells.

FIG. 15 shows the CDC effect of the bispecific antibodies as well as cetuximab.

FIG. 16 shows the ADCC effect on PD-1+ cells.

FIG. 17 shows the CDC effect on PD-1+ cells.

FIG. 18 shows the binding ability of two antibodies to CD28, CTLA-4 and ICOS.

FIG. 19 shows the binding ability of two antibodies to Her2 or Her3.

FIG. 20 shows the melt curves of two bispecific antibodies.

FIG. 21 shows that PD-1-binding of the bispecific antibodies did not lose after incubation in serum for 14 days.

FIG. 22 shows EGFR-binding of the bispecific antibodies slightly lost after incubation in serum for 14 days.

FIG. 23 shows Granzyme B secretion of the cells stimulated by bispecific antibody WBP336B, WBP336C and control antibodies.

FIG. 24 shows that the antibody WBP336B inhibited A431 tumor growth in a mouse model.

FIG. 25 shows the effect of antibodies inhibiting tumor growth in MC38 syngeneic mouse model.

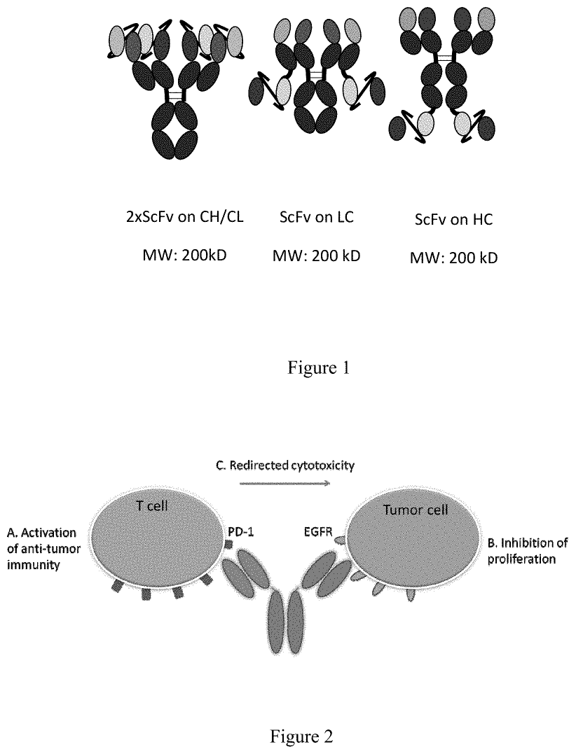

### DETAILED DESCRIPTION

In order that the present invention may be more readily understood, certain terms are first defined. Additional definitions are set forth throughout the detailed description.

The terms “Programmed Death 1”, “Programmed Cell Death 1”, “Protein PD-1”, “PD-1”, “PD1”, “PDCD1”, “hPD-1”, “CD279” and “hPD-F” are used interchangeably, and include variants, isoforms, species homologs of human PD-1, PD-1 of other species, and analogs having at least one common epitope with PD-1.

The term “antibody” as referred to herein includes whole antibodies and any antigen-binding fragment (i.e., “antigen-binding portion”) or single chains thereof. An “antibody” refers to a protein comprising at least two heavy (H) chains and two light (L) chains inter-connected by disulfide bonds, or an antigen-binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The CDRs in heavy chain are abbreviated as H-CDRs, for example H-CDR1, H-CDR2, H-CDR3, and the CDRs in light chain are abbreviated as L-CDRs, for example L-CDR1, L-CDR2, L-CDR3.

The term “antibody” as used in this disclosure, refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. The term “antibody” also includes antibody fragments such as scFv, dAb, and other antibody fragments that retain antigen-binding function, i.e., the ability to bind PD-1 and EGFR specifically. Typically, such fragments would comprise an antigen-binding fragment.

An antigen-binding fragment typically comprises an antibody light chain variable region (VL) and an antibody heavy chain variable region (VH), however, it does not necessarily have to comprise both. For example, a so-called Fd antibody fragment consists only of a VH domain and CH1 domain, but still retains some antigen-binding function of the intact antibody.

The term “cross-reactivity” refers to binding of an antigen fragment described herein to the same target molecule in human, monkey, and/or murine (mouse or rat). Thus, “cross-reactivity” is to be understood as an interspecies reactivity to the same molecule X expressed in different species, but not to a molecule other than X. Cross-species specificity of a monoclonal antibody recognizing e.g. human PD-1, to monkey, and/or to a murine (mouse or rat) PD-1, can be determined, for instance, by FACS analysis.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

The terms “treatment” and “therapeutic method” refer to both therapeutic treatment and prophylactic/preventative measures. Those in need of treatment may include individuals already having a particular medical disorder as well as those who may ultimately acquire the disorder.

The terms “conservative modifications” i.e., nucleotide and amino acid sequence modifications which do not significantly affect or alter the binding characteristics of the antibody encoded by the nucleotide sequence or containing the amino acid sequence. Such conservative sequence modifications include nucleotide and amino acid substitutions, additions and deletions. Modifications can be introduced into the sequence by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The experimental methods in the following examples are conventional methods, unless otherwise specified.

### EXAMPLES

### Example 1: Research Materials Preparation

### 1. Generation of Antigens and Benchmark Antibodies

### 1.1 Generate Soluble Antigens

DNA sequences encoding the extracellular domain sequence of human EGFR (Uniport No.: P00533), human PD-1 (Uniport No.: Q15116), mouse PD-1 (Uniport No.: Q02242), human PD-L1 (Uniport No.: Q9NZQ7), mouse PD-L1 (Uniport No.: Q9EP73) were synthesized in Sangon Biotech (Shanghai, China), and then subcloned into modified pcDNA3.3 expression vectors with different tag (such as 6×his, human Fc, or mouse Fc) in C-terminal.

Expi293 cells (Invitrogen-A14527) were transfected with the purified expression vector pcDNA3.3. Cells were cultured for 5 days and supernatant was collected for protein purification using Ni-NTA column (GE Healthcare, 175248) or Protein A column (GE Healthcare, 175438) or Protein G column (GE Healthcare, 170618). The obtained human EGFR, human PD-1, mouse PD-1, human PD-L1, mouse PD-L1 were QC'ed by SDS-PAGE and SEC, and then stored at −80° C.

### 1.2 Generate Benchmark (BMK) Antibodies

DNA sequence encoding the variable region of anti-EGFR antibody, cetuximab (WBP336-BMK1) was synthesized in Sangon Biotech (Shanghai, China), and then subcloned into modified pcDNA3.3 expression vectors with constant region of human IgG1 or human IgG4 (S228P). Anti-PD-1 antibody W3052-R2-2E5-uIgG4k was generated in house after immunizing rats with human PD-1 and mouse PD-1 and was converted to IgG4(S228P) format.

The plasmid containing VH and VL gene were co-transfected into Expi293 cells. Then the cells were cultured for 5 days and supernatant was collected for protein purification using Protein A column (GE Healthcare, 175438) or Protein G column (GE Healthcare, 170618). The obtained antibodies were evaluated using SDS-PAGE and SEC, and then stored at −80° C.

### 2. Cell Pool/Line Generation

### 2.1 Generate Target-Expressing Cell Lines

Lipofectamine 2000 was used to transfect CHO-S or 293F cells with the expression vector containing gene encoding full length human PD-1 or mouse PD-1. Cells were cultured in medium containing proper selection markers. Human PD-1 high expression stable cell line (WBP305.CHO-S.hPro1.C6) and mouse PD-1 high expression stable cell line (WBP305.293F.mPro1.B4) were obtained by limiting dilution.

The genes of human EGFR, human EGFRvIII, and Macaca fascicularis EGFR were respectively inserted into expression vector pcDNA 3.3. The plasmids were then transfected to CHO-K1 cells respectively, as described below. Briefly, one day prior to transfection, 5×10⁵ CHO-K1 cells were plated into one well of 6-well tissue culture plate and incubated at 5% CO₂ and 37° C. The cells were fed with 3 ml of fresh non-selective media (F12-K, 10% FBS). Transfection reagents were prepared in a 1.5 mL tube, including 4 μg of DNA was mixed with 10 μg of Lipofectamine 2000 to make the final volume 200 μL in Opti-MEM medium. The solution in the tube pipette was added to the cells drop by drop. 6-8 hours after transfection, cells were washed with PBS and feed with 3 ml of fresh non-selective media. Expressing cells were harvested with trypsin 24-48 hours post-transfection and plated to T75 flask in selective media (F12-K, 10% FBS, 10 μg/ml Blasticidin). After two or three passages of selection, the cells were enriched by an anti-EGFR antibody tagged with phycoerythrin (PE) and Anti-PE Microbeads (Miltenyi-013-048-801). Stable single cell clones were isolated by limited dilution and screened by FACS using anti-EGFR antibodies.

### 2.2 Obtain and Culture Target-Expressing Tumor Lines

A431 was purchased from ATCC (ATCC number: CRL-1555) and cultured in DMEM media with 10% fetal bovine serum (FBS). The cells were incubated at 37° C., 5% CO₂ incubator with routine subculturing. For long term storage, the cells were frozen in complete growth medium supplemented with 5% (v/v) DMSO and stored in liquid nitrogen vapor phase.

### Example 2: Bispecific Antibody Generation

### 1. Construct Expression Vectors

Construction of bispecific antibodies: DNA sequence encoding scFv (VH-(G4S)₃-VL) of anti-EGFR antibody with human kappa light chain on the C-terminal was cloned into modified pcDNA3.3 expression vector; DNA sequence encoding scFv (VH-(G4S)₃-VL) of anti-PD1 antibody with the constant region of human IgG4 (S228P) heavy chain on the C-terminal was cloned into modified pcDNA3.3 expression vector.

### 2. Optimize Bispecific Antibodies (Linker and Orientation Etc)

Different from the original construction, the orientation of bispecific antibodies was optimized. DNA sequence encoding scFv (VL-(G4S)₃-VH) of anti-EGFR antibody with human kappa light chain on the C-terminal was cloned into modified pcDNA3.3 expression vector; DNA sequence encoding scFv (VL-(G4S)₃-VH) of anti-PD-1 antibody with the constant region of human IgG4 (S228P) heavy chain on the C-terminal was cloned into modified pcDNA3.3 expression vector.

Two potential glycosylation sites were identified on the variable region of anti-EGFR antibody cetuximab: one is located on the FR2 of light chain and another on FR3 of heavy chain. In order to remove these potential N-glycosylation sites located on the variable region of anti-EGFR antibody cetuximab, several mutations were made based on germline sequences on these positions. The RTNGS on LFR2 was mutated to RTDQS or KPDQS. The QSNDT on HFR3 was mutated to QSEDT or RAEDT. Examples of generated antibodies were listed in Table 1.

### 3. Small Scale Transfection, Expression and Purification

Heavy chain and light chain expression plasmids were co-transfected into ExpiCHO cells using ExpiCHO expression system kit (ThermoFisher-A29133) according to the manufacturer's instructions. Ten days after transfection, the supernatants were collected and used for protein purification using Protein A column (GE Healthcare-17543802) and further size exclusion chromatography (GE Healthcare-17104301). Antibody concentration was measured by Nano Drop. The purity of proteins was evaluated by SDS-PAGE and HPLC-SEC. Two Bispecific antibodies, i.e. W336-T1U2.G10-4.uIgG4.SP(dk) and W336-T1U3.G10-4.uIgG4.SP(dk) were obtained after expression and purification.

### 4. Produce Bispecific Antibody for In Vivo Studies (Including Endotoxin Control and Test)

The pair of WBP336B (W336-T1U2.G10-4.uIgG4.SP(dk)) or WBP336C (W336-T1U3.G10-4.uIgG4.SP(dk)) expression plasmids were co-transfected into ExpiCHO cells using ExpiCHO expression system kit (ThermoFisher-A29133) according to the manufacturer's instructions. Ten days after transfection, the supernatants were collected and used for protein purification using Protein A column (GE Healthcare-17543802) and further size exclusion chromatography (GE Healthcare-17104301) under endotoxin control condition. The endotoxin level was confirmed by using endotoxin detection kit (GenScript-L00350), and the endotoxin level of two Bispecific antibodies was both less than 10 EU/mg. The purity of proteins was evaluated by SDS-PAGE and HPLC-SEC.

### 5. Results

### 5.1 Sequence of Lead Candidates

The sequences of antibody leads are listed in the Table 3 and the CDRs are listed in Table 4.

TABLE 3
Deduced amino acid sequences of bispecific antibodies

| | | SEQ ID | |
|---|---|---|---|
| Clone ID | | NO | Amino acid sequence |
| WBP336B = | Second | 19 | DVVMTQSPLSLPVTLGQPASISCRSSQSLLDSDGGTYL |
| W336- | polypeptide | | YWFQQRPGQSPRRLIYLVSTLGSGVPDRFSGSGSGTDF |
| T1U2.G10- | (underlined | | TLKISRVEAEDVGVYYCMQLTHWPYTFGQGTKLEIKGG |
| 4.uhIgG4.SP | VL and VH, | | GGSGGGGSGGGGSQVQLVQSGAEVKKPGSSVKVSCKAS |
| (dK) | anti-PD-1) | | GFTFTTYYISWVRQAPGQGLEYLGYINMGSGGTNYNEK |
| | | | FKGRVTITADKSTSTAYMELSSLRSEDTAVYYCAILGY |
| | | | FDYWGQGTMVTVSSASTKGPSVFPLAPCSRSTSESTAA |
| | | | LGCLVKDYFPEPVTVSWNSGALTSGVHTFPAVLQSSGL |
| | | | YSLSSVVTVPSSSLGTKTYTCNVDHKPSNTKVDKRVES |
| | | | KYGPPCPPCPAPEFLGGPSVFLFPPKPKDTLMISRTPE |
| | | | VTCVVVDVSQEDPEVQFNWYVDGVEVHNAKTKPREEQF |
| | | | NSTYRVVSVLTVLHQDWLNGKEYKCKVSNKGLPSSIEK |
| | | | TISKAKGQPREPQVYTLPPSQEEMTKNQVSLTCLVKGF |
| | | | YPSDIAVEWESNGQPENNYKTTPPVLDSDGSFFLYSRL |
| | | | TVDKSRWQEGNVFSCSVMHEALHNHYTQKSLSLSLG |
| | First | 20 | DILLTQSPVILSVSPGERVSFSCRASQSIGTNIHWYQQ |
| | polypeptide | | RTNGSPRLLIKYASESISGIPSRFSGSGSGTDFTLSIN |
| | (underlined | | SVESEDIADYYCQQNNNWPTTFGAGTKLELKGGGGSGG |
| | VL and VH, | | GGSGGGGSQVQLKQSGPGLVQPSQSLSITCTVSGFSLT |
| | anti-EGFR) | | NYGVHWVRQSPGKGLEWLGVIWSGGNTDYNTPFTSRLS |
| | | | INKDNSKSQVFFKMNSLQSNDTAIYYCARALTYYDYEF |
| | | | AYWGQGTLVTVSARTVAAPSVFIFPPSDEQLKSGTASV |
| | | | VCLLNNFYPREAKVQWKVDNALQSGNSQESVTEQDSKD |
| | | | STYSLSSTLTLSKADYEKHKVYACEVTHQGLSSPVTKS |
| | | | FNRGEC |
| WBP336C = | Second | 21 | DVVMTQSPLSLPVTLGQPASISCRSSQSLLDSDGGTYL |
| W336- | polypeptide | | YWFQQRPGQSPRRLIYLVSTLGSGVPDRFSGSGSGTDF |
| T1U3.G10- | (underlined | | TLKISRVEAEDVGVYYCMQLTHWPYTFGQGTKLEIKGG |
| 4.uhIgG4.SP | VL and VH, | | GGSGGGGSGGGGSQVQLVQSGAEVKKPGSSVKVSCKAS |
| (dK) | anti-PD-1) | | GFTFTTYYISWVRQAPGQGLEYLGYINMGSGGTNYNEK |
| | | | FKGRVTITADKSTSTAYMELSSLRSEDTAVYYCAIIGY |
| | | | FDYWGQGTMVTVSSASTKGPSVFPLAPCSRSTSESTAA |
| | | | LGCLVKDYFPEPVTVSWNSGALTSGVHTFPAVLQSSGL |
| | | | YSLSSVVTVPSSSLGTKTYTCNVDHKPSNTKVDKRVES |
| | | | KYGPPCPPCPAPEFLGGPSVFLFPPKPKDTLMISRTPE |
| | | | VTCVVVDVSQEDPEVQFNWYVDGVEVHNAKTKPREEQF |
| | | | NSTYRVVSVLTVLHQDWLNGKEYKCKVSNKGLPSSIEK |
| | | | TISKAKGQPREPQVYTLPPSQEEMTKNQVSLTCLVKGF |
| | | | YPSDIAVEWESNGQPENNYKTTPPVLDSDGSFFLYSRL |
| | | | TVDKSRWQEGNVFSCSVMHEALHNHYTQKSLSLSLG |
| | First | 20 | DILLTQSPVILSVSPGERVSFSCRASQSIGTNIHWYQQ |
| | polypeptide | | RTNGSPRLLIKYASESISGIPSRFSGSGSGTDFTLSIN |
| | (underlined | | SVESEDIADYYCQQNNNWPTTFGAGTKLELKGGGGSGG |
| | VL and VH, | | GGSGGGGSQVQLKQSGPGLVQPSQSLSITCTVSGFSLT |
| | anti-EDFR) | | NYGVHWVRQSPGKGLEWLGVIWSGGNTDYNTPFTSRLS |
| | | | INKDNSKSQVFFKMNSLQSNDTAIYYCARALTYYDYEF |
| | | | AYWGQGTLVTVSARTVAAPSVFIFPPSDEQLKSGTASV |
| | | | VCLLNNFYPREAKVQWKVDNALQSGNSQESVTEQDSKD |
| | | | STYSLSSTLTLSKADYEKHKVYACEVTHQGLSSPVTKS |
| | | | FNRGEC |
| WBP336D | Second | 19 | DVVMTQSPLSLPVTLGQPASISCRSSQSLLDSDGGTYL |
| | polypeptide | | YWFQQRPGQSPRRLIYLVSTLGSGVPDRFSGSGSGTDF |
| | (underlined | | TLKISRVEAEDVGVYYCMQLTHWPYTFGQGTKLEIKGG |
| | VL and VH, | | GGSGGGGSGGGGSQVQLVQSGAEVKKPGSSVKVSCKAS |
| | anti-PD-1) | | GFTFTTYYISWVRQAPGQGLEYLGYINMGSGGTNYNEK |
| | | | FKGRVTITADKSTSTAYMELSSLRSEDTAVYYCAILGY |
| | | | FDYWGQGTMVTVSSASTKGPSVFPLAPCSRSTSESTAA |
| | | | LGCLVKDYFPEPVTVSWNSGALTSGVHTFPAVLQSSGL |
| | | | YSLSSVVTVPSSSLGTKTYTCNVDHKPSNTKVDKRVES |
| | | | KYGPPCPPCPAPEFLGGPSVFLFPPKPKDTLMISRTPE |
| | | | VTCVVVDVSQEDPEVQFNWYVDGVEVHNAKTKPREEQF |
| | | | NSTYRVVSVLTVLHQDWLNGKEYKCKVSNKGLPSSIEK |
| | | | TISKAKGQPREPQVYTLPPSQEEMTKNQVSLTCLVKGF |
| | | | YPSDIAVEWESNGQPENNYKTTPPVLDSDGSFFLYSRL |
| | | | TVDKSRWQEGNVFSCSVMHEALHNHYTQKSLSLSLG |
| | First | 22 | DILLTQSPVILSVSPGERVSFSCRASQSIGTNIHWYQQ |
| | polypeptide | | RTDQSPRLLIKYASESISGIPSRFSGSGSGTDFTLSIN |
| | (underlined | | SVESEDIADYYCQQNNNWPTTFGAGTKLELKGGGGSGG |
| | VL and VH, | | GGSGGGGSQVQLKQSGPGLVQPSQSLSITCTVSGFSLT |
| | anti-EGFR) | | NYGVHWVRQSPGKGLEWLGVIWSGGNTDYNTPFTSRLS |
| | | | INKDNSKSQVFFKMNSLQSEDTAIYYCARALTYYDYEF |
| | | | AYWGQGTLVTVSARTVAAPSVFIFPPSDEQLKSGTASV |
| | | | VCLLNNFYPREAKVQWKVDNALQSGNSQESVTEQDSKD |
| | | | STYSLSSTLTLSKADYEKHKVYACEVTHQGLSSPVTKS |
| | | | FNRGEC |
| WBP336E | Second | 19 | DVVMTQSPLSLPVTLGQPASISCRSSQSLLDSDGGTYL |
| | polypeptide | | YWFQQRPGQSPRRLIYLVSTLGSGVPDRFSGSGSGTDF |
| | (underlined | | TLKISRVEAEDVGVYYCMQLTHWPYTFGQGTKLEIKGG |
| | VL and VH, | | GGSGGGGSGGGGSQVQLVQSGAEVKKPGSSVKVSCKAS |
| | anti-PD-1) | | GFTFTTYYISWVRQAPGQGLEYLGYINMGSGGTNYNEK |
| | | | FKGRVTITADKSTSTAYMELSSLRSEDTAVYYCAILGY |
| | | | FDYWGQGTMVTVSSASTKGPSVFPLAPCSRSTSESTAA |
| | | | LGCLVKDYFPEPVTVSWNSGALTSGVHTFPAVLQSSGL |
| | | | YSLSSVVTVPSSSLGTKTYTCNVDHKPSNTKVDKRVES |
| | | | KYGPPCPPCPAPEFLGGPSVFLFPPKPKDTLMISRTPE |
| | | | VTCVVVDVSQEDPEVQFNWYVDGVEVHNAKTKPREEQF |
| | | | NSTYRVVSVLTVLHQDWLNGKEYKCKVSNKGLPSSIEK |
| | | | TISKAKGQPREPQVYTLPPSQEEMTKNQVSLTCLVKGF |
| | | | YPSDIAVEWESNGQPENNYKTTPPVLDSDGSFFLYSRL |
| | | | TVDKSRWQEGNVFSCSVMHEALHNHYTQKSLSLSLG |
| | First | 23 | DILLTQSPVILSVSPGERVSFSCRASQSIGTNIHWYQQ |
| | polypeptide | | KPDQSPRLLIKYASESISGIPSRFSGSGSGTDFTLSIN |
| | (underlined | | SVESEDIADYYCQQNNNWPTTFGAGTKLELKGGGGSGG |
| | VL and VH, | | GGSGGGGSQVQLKQSGPGLVQPSQSLSITCTVSGFSLT |
| | anti-EGFR) | | NYGVHWVRQSPGKGLEWLGVIWSGGNTDYNTPFTSRLS |
| | | | INKDNSKSQVFFKMNSLRAEDTAIYYCARALTYYDYEF |
| | | | AYWGQGTLVTVSARTVAAPSVFIFPPSDEQLKSGTASV |
| | | | VCLLNNFYPREAKVQWKVDNALQSGNSQESVTEQDSKD |
| | | | STYSLSSTLTLSKADYEKHKVYACEVTHQGLSSPVTKS |
| | | | FNRGEC |
| WBP336F | Second | 21 | DVVMTQSPLSLPVTLGQPASISCRSSQSLLDSDGGTYL |
| | polypeptide | | YWFQQRPGQSPRRLIYLVSTLGSGVPDRFSGSGSGTDF |
| | (underlined | | TLKISRVEAEDVGVYYCMQLTHWPYTFGQGTKLEIKGG |
| | VL and VH, | | GGSGGGGSGGGGSQVQLVQSGAEVKKPGSSVKVSCKAS |
| | anti-PD-1) | | GFTFTTYYISWVRQAPGQGLEYLGYINMGSGGTNYNEK |
| | | | FKGRVTITADKSTSTAYMELSSLRSEDTAVYYCAIIGY |
| | | | FDYWGQGTMVTVSSASTKGPSVFPLAPCSRSTSESTAA |
| | | | LGCLVKDYFPEPVTVSWNSGALTSGVHTFPAVLQSSGL |
| | | | YSLSSVVTVPSSSLGTKTYTCNVDHKPSNTKVDKRVES |
| | | | KYGPPCPPCPAPEFLGGPSVFLFPPKPKDTLMISRTPE |
| | | | VTCVVVDVSQEDPEVQFNWYVDGVEVHNAKTKPREEQF |
| | | | NSTYRVVSVLTVLHQDWLNGKEYKCKVSNKGLPSSIEK |
| | | | TISKAKGQPREPQVYTLPPSQEEMTKNQVSLTCLVKGF |
| | | | YPSDIAVEWESNGQPENNYKTTPPVLDSDGSFFLYSRL |
| | | | TVDKSRWQEGNVFSCSVMHEALHNHYTQKSLSLSLG |
| | First | 22 | DILLTQSPVILSVSPGERVSFSCRASQSIGTNIHWYQQ |
| | polypeptide | | RTDQSPRLLIKYASESISGIPSRFSGSGSGTDFTLSIN |
| | (underlined | | SVESEDIADYYCQQNNNWPTTFGAGTKLELKGGGGSGG |
| | VL and VH, | | GGSGGGGSQVQLKQSGPGLVQPSQSLSITCTVSGFSLT |
| | anti-EGFR) | | NYGVHWVRQSPGKGLEWLGVIWSGGNTDYNTPFTSRLS |
| | | | INKDNSKSQVFFKMNSLQSEDTAIYYCARALTYYDYEF |
| | | | AYWGQGTLVTVSARTVAAPSVFIFPPSDEQLKSGTASV |
| | | | VCLLNNFYPREAKVQWKVDNALQSGNSQESVTEQDSKD |
| | | | STYSLSSTLTLSKADYEKHKVYACEVTHQGLSSPVTKS |
| | | | FNRGEC |
| WBP336G | Second | 21 | DVVMTQSPLSLPVTLGQPASISCRSSQSLLDSDGGTYL |
| | polypeptide | | YWFQQRPGQSPRRLIYLVSTLGSGVPDRFSGSGSGTDF |
| | (underlined | | TLKISRVEAEDVGVYYCMQLTHWPYTFGQGTKLEIKGG |
| | VL and VH, | | GGSGGGGSGGGGSQVQLVQSGAEVKKPGSSVKVSCKAS |
| | anti-PD-1) | | GFTFTTYYISWVRQAPGQGLEYLGYINMGSGGTNYNEK |
| | | | FKGRVTITADKSTSTAYMELSSLRSEDTAVYYCAIIGY |
| | | | FDYWGQGTMVTVSSASTKGPSVFPLAPCSRSTSESTAA |
| | | | LGCLVKDYFPEPVTVSWNSGALTSGVHTFPAVLQSSGL |
| | | | YSLSSVVTVPSSSLGTKTYTCNVDHKPSNTKVDKRVES |
| | | | KYGPPCPPCPAPEFLGGPSVFLFPPKPKDTLMISRTPE |
| | | | VTCVVVDVSQEDPEVQFNWYVDGVEVHNAKTKPREEQF |
| | | | NSTYRVVSVLTVLHQDWLNGKEYKCKVSNKGLPSSIEK |
| | | | TISKAKGQPREPQVYTLPPSQEEMTKNQVSLTCLVKGF |
| | | | YPSDIAVEWESNGQPENNYKTTPPVLDSDGSFFLYSRL |
| | | | TVDKSRWQEGNVFSCSVMHEALHNHYTQKSLSLSLG |
| | First | 23 | DILLTQSPVILSVSPGERVSFSCRASQSIGTNIHWYQQ |
| | polypeptide | | KPDQSPRLLIKYASESISGIPSRFSGSGSGTDFTLSIN |
| | (underlined | | SVESEDIADYYCQQNNNWPTTFGAGTKLELKGGGGSGG |
| | VL and VH, | | GGSGGGGSQVQLKQSGPGLVQPSQSLSITCTVSGFSLT |
| | anti-EGFR) | | NYGVHWVRQSPGKGLEWLGVIWSGGNTDYNTPFTSRLS |
| | | | INKDNSKSQVFFKMNSLRAEDTAIYYCARALTYYDYEF |
| | | | AYWGQGTLVTVSARTVAAPSVFIFPPSDEQLKSGTASV |
| | | | VCLLNNFYPREAKVQWKVDNALQSGNSQESVTEQDSKD |
| | | | STYSLSSTLTLSKADYEKHKVYACEVTHQGLSSPVTKS |
| | | | FNRGEC |

TABLE 4
CDRs of WBP336B and WBP336C

| | | SEQ | |
|---|---|---|---|
| | | ID | |
| Clone ID | | NO | Amino acid sequence |
| WBP336B = | Anti-EGFR: | 6 | GFSLTNYGVH |
| W336- | HCDR1 | | |
| T1U2.G10- | Anti-EGFR: | 7 | VIWSGGNTDYNTPFTS |
| 4.uhIgG4.SP | HCDR2 | | |
| (dK) | Anti-EGFR: | 8 | ALTYYDYEFAY |
| | HCDR3 | | |
| | Anti-EGFR: | 9 | RASQSIGTNIH |
| | LCDR1 | | |
| | Anti-EGFR: | 10 | YASESIS |
| | LCDR2 | | |
| | Anti-EGFR: | 11 | QQNNNWPTT |
| | LCDR3 | | |
| | Anti-PD-1: | 12 | GFTFTTYYIS |
| | HCDR1 | | |
| | Anti-PD-1: | 13 | YINMGSGGTNYNEKFKG |
| | HCDR2 | | |
| | Anti-PD-1: | 14 | LGYFDY |
| | HCDR3 | | |
| | Anti-PD-1: | 15 | RSSQSLLDSDGGTYLY |
| | LCDR1 | | |
| | Anti-PD-1: | 16 | LVSTLGS |
| | LCDR2 | | |
| | Anti-PD-1: | 17 | MQLTHWPYT |
| | LCDR3 | | |
| WBP336C = | Anti-EGFR: | 6 | GFSLTNYGVH |
| W336- | HCDR1 | | |
| T1U3.G10- | Anti-EGFR: | 7 | VIWSGGNTDYNTPFTS |
| 4.uhIgG4.SP | HCDR2 | | |
| (dK) | Anti-EGFR: | 8 | ALTYYDYEFAY |
| | HCDR3 | | |
| | Anti-EGFR: | 9 | RASQSIGTNIH |
| | LCDR1 | | |
| | Anti-EGFR: | 10 | YASESIS |
| | LCDR2 | | |
| | Anti-EGFR: | 11 | QQNNNWPTT |
| | LCDR3 | | |
| | Anti-PD-1: | 12 | GFTFTTYYIS |
| | HCDR1 | | |
| | Anti-PD-1: | 13 | YINMGSGGTNYNEKFKG |
| | HCDR2 | | |
| | Anti-PD-1: | 18 | IGYFDY |
| | HCDR3 | | |
| | Anti-PD-1: | 15 | RSSQSLLDSDGGTYLY |
| | LCDR1 | | |
| | Anti-PD-1: | 16 | LVSTLGS |
| | LCDR2 | | |
| | Anti-PD-1: | 17 | MQLTHWPYT |
| | LCDR3 | | |

### Example 3: Possible Mechanisms of Targeting EGFR and PD-1

We have proposed three possible mechanisms that a bispecific antibody against EGFR and PD-1 can improve anti-tumor effects (FIG. 2). First, the antibody can block EGFR pathway, inhibiting tumor proliferation, migration etc. Second, the antibody can block PD-1 pathway, resuming or improving the anti-tumor function of T cells. Lastly, the antibody can bridge tumor cells and T cells, likely improving the anti-tumor effect. This could also help to enrich anti-PD-1 antibody in a tumor microenvironment.

### Example 4: In Vitro Characterization

### 1. Protein Analytics

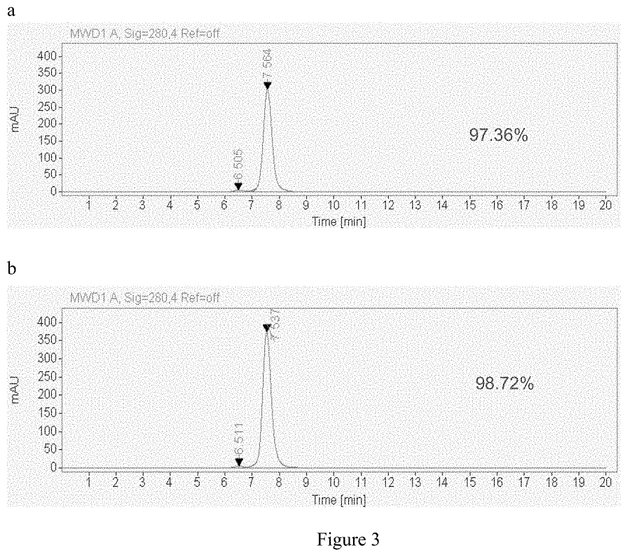

The two lead antibodies were expressed from ExpiCHO cells, and then purified using Protein A chromatography and size-exclusion chromatography. As shown in Table 5 and FIG. 3, the two antibodies had reasonable expression level and high purity.

TABLE 5
Purification of bispecific antibodies

| | Conc. | Amount | Purity | Yield |
|---|---|---|---|---|
| Protein Name | (mg/ml) | (mg) | (%) | (mg/l) |
| WBP336B = W336-T1U2. | 1.6 | 1.9 | 97.36% | 40.5 |
| G10-4.uIgG4.SP (dK) | | | | |
| WBP336C = W336-T1U3. | 2.0 | 2.4 | 98.72% | 35.8 |
| G10-4.uIgG4.SP (dK) | | | | |

2a. EGFR- or PD-1-Binding (ELISA and FACS)

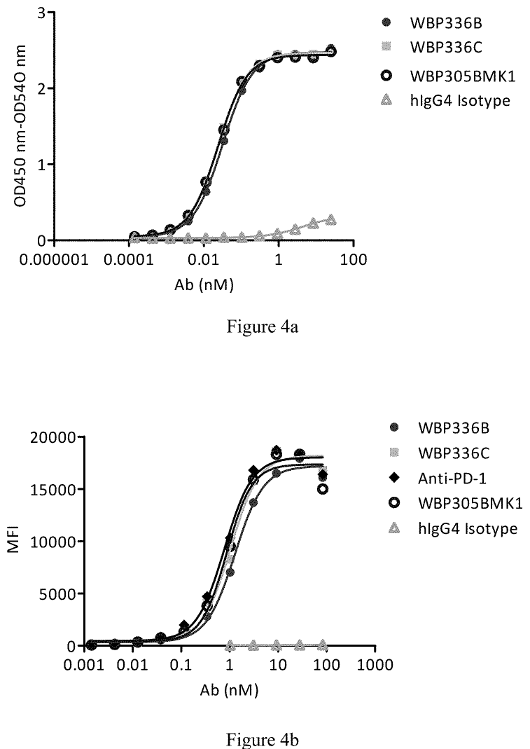

Two antibody leads were characterized in their binding to PD-1 in both ELISA (FIG. 4A) and FACS (FIG. 4B). For ELISA binding, non-tissue culture treated flat-bottom 96-well plates were pre-coated with 0.5 μg/ml in house made human PD-1 protein WBP305-hPro1.ECD.mFc overnight at 4° C. After 2% BSA blocking, 100 μL 3-fold titrated Abs from 25 nM to 0.0001 nM Abs were pipetted into each well and incubated for 1 hour at ambient temperature. Following removal of the unbound substances, HRP-labeled goat anti-human IgG were added to the wells and incubated for 1 hour. The color was developed by dispensing 100 μL TMB substrate, and then stopped by 100 μL 2N HCl. The absorbance was read at 450 nm using a Microplate Spectrophotometer.

For FACS binding, engineered human PD-1 expressing cells WBP305.CHO-S.hPro1.C6 were seeded at 1×10⁵ cells/well in U-bottom 96-well plates. 3-Fold titrated Abs from 83.3 nM to 0.001 nM were added to the cells. Plates were incubated at 4° C. for 1 hour. After wash, PE-labeled goat anti-human antibody was added to each well and the plates were incubated at 4° C. for 1 hour. The binding of the antibodies onto the cells was tested by flow cytometry and the mean fluorescence intensity (MFI) was analyzed by FlowJo.

Binding of the bispecific antibodies to EGFR expressing cells was determined by flow cytometry. Briefly, 1×10⁵ A431 (EGFR+) cells or cynomolgus monkey EGFR over-expressed stable cell line (WBP562-CHOK1.cPro1.H6) were incubated for 60 minutes at 4° C. with serial dilutions of EGFR×PD-1 bispecific or hIgG4 isotype control antibodies. After washing twice with cold PBS supplemented with 1% bovine serum albumin (wash buffer), cell surface bound antibody was detected by incubating the cells with Fluorescence-labeled anti-human IgG antibody for 30 minutes at 4° C. Cells were washed twice in the same buffer and the mean fluorescence (MFI) of stained cells was measured using a FACS Canto II cytometer (BD Biosciences). Wells containing no antibody or secondary antibody only were used to establish background fluorescence. Four-parameter non-linear regression analysis was used to obtain EC₅₀ values for cell binding using GraphPad Prism software.

WBP336B (EC₅₀=0.032 nM) and WBP336C (EC₅₀=0.024 nM) bound to PD-1 comparable with their parental antibody (EC₅₀=0.031 nM) or WBP305-BMK1 (EC₅₀=0.024 nM). FACS was used to test these antibodies binding on cell surface PD-1. WBP336B and WBP336C bound to PD-1 positive cells with EC₅₀ of 1.29 and 1.05 nM, respectively, slightly higher than the EC₅₀ of their parental antibody (0.78 nm) and BMK1 (0.87 nM).

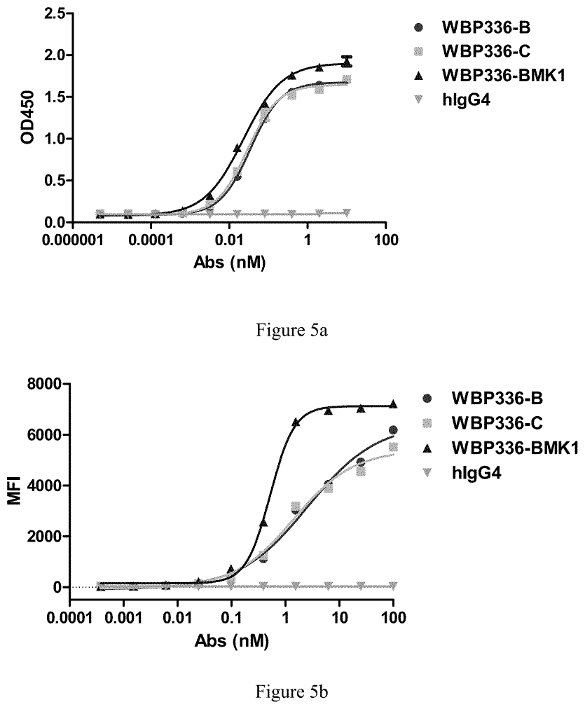

The similar assays were used to test the antibody-binding to EGFR (FIGS. 5A and 5B). 96-well ELISA plates (Nunc MaxiSorp, ThermoFisher) are coated overnight at 4° C. with 0.5 g/ml antigen (EGFR-ECD, W562-hPro1.ECD.his (sino)) in Carbonate-bicarbonate buffer. After a 1 hour blocking step with 2% (w/v) bovine serum albumin (Pierce) dissolved in PBS, serial dilutions of the different EGFR×PD-1 bispecific antibodies in PBS containing 2% bovine serum albumin are incubated on the plates for 2 hours at room temperature. Following the incubation, plates are washed three times with 300 μL per well of PBS containing 0.5% (v/v) Tween 20. 100 ng/ml Goat-anti-human IgG Fc-HRP (Bethyl, #A80-304P) is added and incubated on the plates for 1 hour at room temperature. After washing six times with 300 μL per well of PBS containing 0.5% (v/v) Tween 20, Tetramethylbenzidine (TMB) Substrate (Sigma-860336-5G) is added for the detection. The reaction is stopped after approximate 8 minutes through the addition of 100 μL per well of 2 M HCl. The absorbance of the wells is measured at 450 nm with a multiwall plate reader (SpectraMax® M5e).

In ELISA, WBP336B and WBP336C bound to human EGFR with EC₅₀ of 0.035 and 0.029 nM respectively, comparable to Cetuximab binding to EGFR with EC₅₀=0.023 nM. The difference between WBP336B/C and Cetuximab is more significant in binding on cell surface EGFR. Using A431 cells as target cells, the binding of WBP336B and WBP336C bound to A431 EC₅₀ of 2.6 and 1.4 nM, whereas the Cetuximab bound to EGFR with EC₅₀=0.5 nM.

2b. EGFR- and PD-1-Dual Binding (ELISA and FACS)

In order to test whether the bispecific antibodies could bind to both PD-1 and EGFR, an ELISA assay was developed as below. A 96-well ELISA plate (Nunc MaxiSorp, ThermoFisher) was coated overnight at 4° C. with 0.5 μg/ml antigen-1 (EGFR-ECD, W562-hPro1.ECD.his (sino)) in carbonate-bicarbonate buffer. After a 1 hour blocking step with 2% (w/v) bovine serum albumin (Pierce) dissolved in PBS, serial dilutions of the different EGFR×PD-1 bispecific antibodies in PBS containing 2% bovine serum albumin are incubated on the plates for 1 hour at room temperature. Following the incubation, plates are washed three times with 300 μL per well of PBS containing 0.5% (v/v) Tween 20. 0.1 μg/ml antigen-2 (PD-1-ECD, WBP305-hPro1.ECD.hFc.Biotin) was added to plates and incubation 1 hour. After washing the plates three times, Streptavidin-RP (Invitrogen, #SNN1004) (1:25000 diluted) is added and incubated on the plates for 1 hour at room temperature. After washing six times with 300 μL per well of PBS containing 0.5% (v/v) Tween 20, Tetramethylbenzidine (TMB) Substrate (Sigma-860336-5G) is added for the detection. The reaction is stopped after approximate 10 minutes through the addition of 100 μL per well of 2 M HCl. The absorbance of the wells is measured at 450 nm with a multiwall plate reader (SpectraMax® M5e).

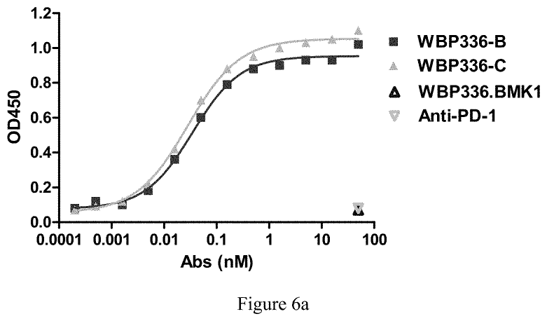

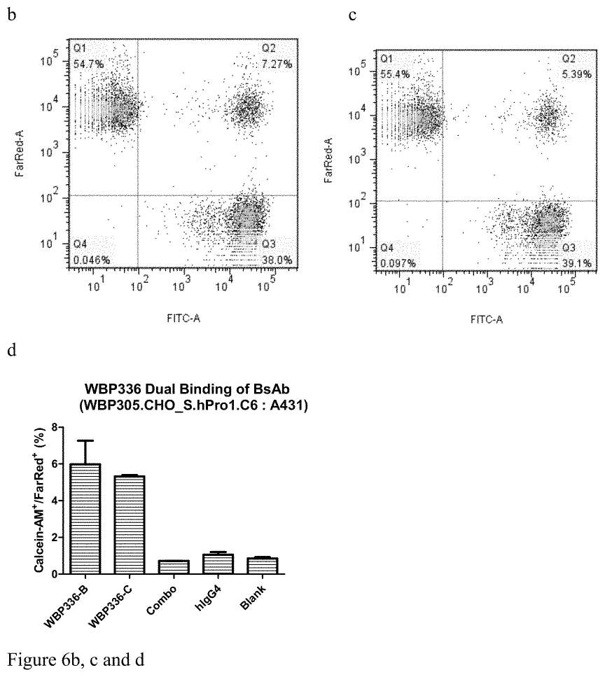

As shown in FIG. 6a, the two antibodies were able to bind both targets, with EC₅₀=0.035 nM and 0.028 nM respectively.

The ability of EGFR×PD-1 bispecific antibodies to bridge two target cells was tested by flow cytometry. 1×10⁶/ml EGFR⁺ A431 cells or PD-1⁺ CHOK-S cells were labeled with 50 nM Calcein-AM (Invitrogen-C3099) or 20 nM FarRed (Invitrogen-C34572) respectively, for 30 minutes at 37° C. and washed twice with 1% fetal bovine serum. The cells of each type were resuspended and then mixed to a final concentration of 1×10⁶/ml at the ratio of 1:1. The antibodies were added to the cells followed by gentle mixing and one-hour incubation. Bridging % was calculated as the percentage of events that were simultaneously labeled calcein-AM and FarRed.

As shown in FIG. 6b, c and d, compared with combination of two monospecific antibodies or isotype control antibody, the bispecific antibodies can increase the cell population with both Far-Red and CAlcein-AM staining, demonstrating that the bispecific antibody did bridge two kinds of cells together.

### 3. Cross Species Binding (ELISA/FACS)

As the parental anti-PD-1 antibody was able to bind cynomolgus and murine target, the cross-species binding of the two bispecific antibodies were investigated. Antibodies were detected on their binding to mouse PD-1 in a FACS assay. Briefly, engineered mouse PD-1 expressing cells WBP305.293F.mPro1.B4 were seeded at 1×10⁵ cells/well in U-bottom 96-well plates. 3-Fold titrated Abs from 133.3 nM to 0.06 nM were added to the cells. Plates were incubated at 4° C. for 1 hour. After wash, PE-labeled goat anti-human antibody was added to each well and the plates were incubated at 4° C. for 1 hour. The binding of the antibodies onto the cells was tested by flow cytometry and the mean fluorescence intensity (MFI) was analyzed by FlowJo.

Cynomolgus PD-1-binding ELISA was used to test the antibodies. Briefly, flat-bottom 96-well plates were pre-coated with 0.5 ug/ml in-house made cynomolgus PD-1 protein WBP305-cPro1.ECD.his overnight at 4° C. After 2% BSA blocking, 100 μL 3-fold titrated Abs from 25 nM to 0.0001 nM Abs were pipetted into each well and incubated for 1 hour at ambient temperature. Following removal of the unbound substances, HRP-labeled goat anti-human IgG was added to the wells and incubated for 1 hour. The color was developed by dispensing 100 μL TMB substrate, and then stopped by 100 μL 2N HCl. The absorbance was read at 450 nm using a Microplate Spectrophotometer.

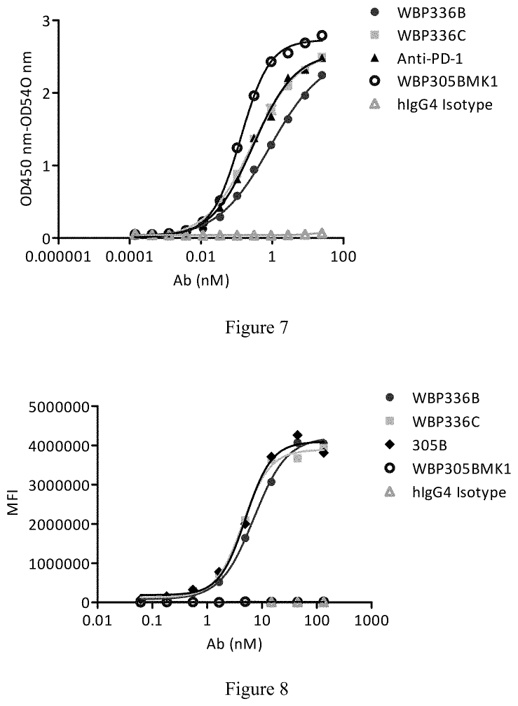

As show in FIG. 7, WBP336C (EC₅₀=0.275 nM) had similar binding to cynomolgus PD-1 with its parental antibody (0.295 nM), whereas WBP336B had reduced binding activity (EC₅₀=0.874 nM). In comparison, WBP305-BMK1 had binding activity with EC₅₀=0.132 nM.

In a FACS assay, the bispecific antibodies were tested binding to murine PD-1. As shown in FIG. 8, WBP336B and WBP336C bound to murine PD-1 with EC₅₀ 7.11 and 4.47 nM respectively, similar to its parental antibody 5.01 nM. In contrast, WBP305-BMK1 did not bind to murine PD-1 at all.

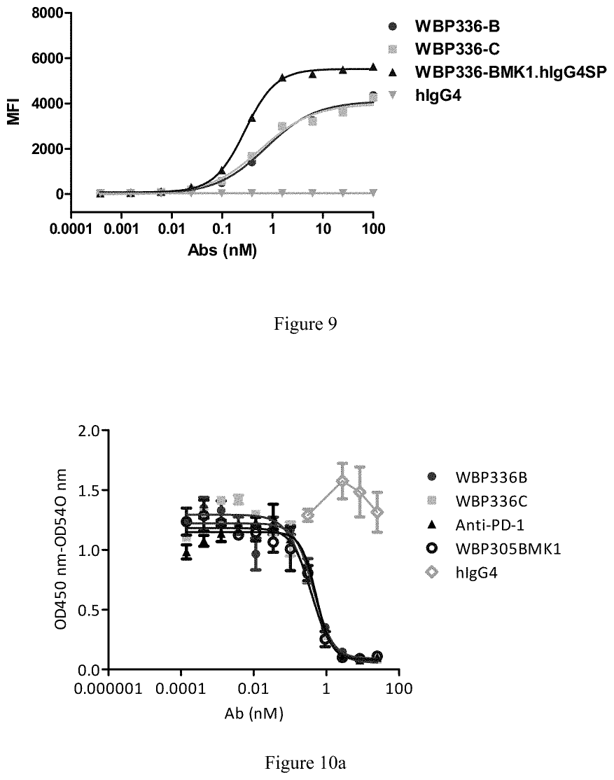

It was reported that cetuximab bound to cynomolgus EGFR but not murine EGFR. Therefore, we only test the bispecific antibodies binding on cynomolgus EGFR. As shown in FIG. 9, WBP336B and WBP336C bound to EGFR with EC₅₀ of 0.75 and 0.59 nM, whereas cetuximab bound with EC₅₀ 0.29 nM.

### 4. Affinity of the Bispecific Antibodies

SPR technology was used to measure the on-rate constant (ka) and off-rate constant (kd) of the antibodies to ECD of EGFR or PD-1. The affinity constant (KD) was consequently determined.

Biacore T200, Series S Sensor Chip CM5, Amine Coupling Kit, and 10×HBS-EP were purchased from GE Healthcare. Goat anti-human IgG Fc antibody was purchased from Jackson ImmunoResearch Lab (catalog number 109-005-098). In immobilization step, the activation buffer was prepared by mixing 400 mM EDC and 100 mM NHS immediately prior to injection. The CM5 sensor chip was activated for 420 s with the activation buffer. 30 μg/mL of goat anti-human IgG Fcγ antibody in 10 mM NaAc (pH 4.5) was then injected to Fc1-Fc4 channels for 200 s at a flow rate of 5 μL/min. The chip was deactivated by 1 M ethanolamine-HCl (GE). Then the antibodies were captured on the chip. Briefly, 4 g/mL antibodies in running buffer (HBS-EP+) was injected individually to Fc3 channel for 30 s at a flow rate of 10 μL/min. Eight different concentrations (20, 10, 5, 2.5, 1.25, 0.625, 0.3125 and 0.15625 nM) of analyte ECD of EGFR or PD-1 and blank running buffer were injected orderly to Fc1-Fc4 channels at a flow rate of 30 μL/min for an association phase of 120 s, followed by 2400 s dissociation phase. Regeneration buffer (10 mM Glycine pH 1.5) was injected at 10 μL/min for 30 s following every dissociation phase.

As shown in Table 6, both WBP336B and WBP336C bound to PD-1 and EGFR with high affinity. They bound to hPD-1 with K_{D} of 8 and 2 nM, higher than that of their parental antibody's 0.65 nM. The high K_{D} mainly contributed by fast kd, whereas ka did not significantly change. Compared with their parental Ab cetuximab, their binding to EGFR did not change.

TABLE 6

| Antigen | Antibody | ka (1/Ms) | kd (1/s) | K_{D} (M) |
|---|---|---|---|---|
| hPD-1.ECD | WBP336B | 1.27E+06 | 9.75E−03 | 7.68E−09 |
| | WBP336C | 1.21E+06 | 2.40E−03 | 1.98E−09 |
| | Parental mAb | 8.03E+05 | 5.19E−04 | 6.47E−10 |
| hEGFR.ECD | WBP336B | 1.30E+06 | 7.16E−04 | 5.49E−10 |
| | WBP336C | 1.36E+06 | 7.41E−04 | 5.45E−10 |
| | Parental mAb | 1.19E+06 | 6.46E−04 | 5.45E−10 |

5. Competition Based Functional Assays (e.g. Ligand Competition Assay)

The functionality of the bispecific antibodies was investigated using different assays.

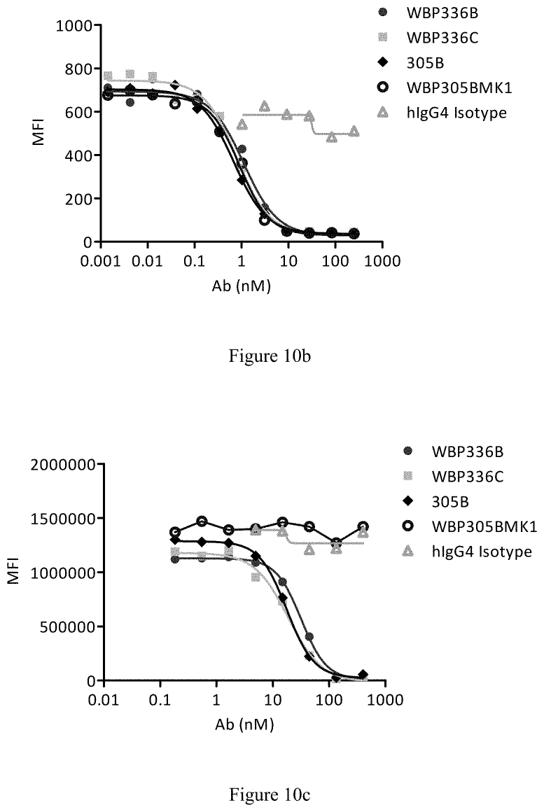

First, the bispecific antibodies were able to block PD-1 binding to PD-L1 in an ELISA-based competition assay, as shown in FIG. 10a. WBP336B and WBP336C showed IC₅₀ of 0.454 nM and 0.352 nM respectively, comparable with their parental Ab 305B (IC₅₀=0.524 nM). The increased potency of bispecific antibodies might due to their larger size than regular IgG, which improved blocking effect by steric hinderance.

A FACS-based competition assay was also performed to evaluate the bispecific antibodies on cell surface PD-1. Briefly, 1×10⁵ A431 (EGFR+) cells were incubated for 60 minutes at 4° C. with serial dilutions of EGFR×PD-1 bispecific or hIgG4 isotype control antibodies and 0.1 g/ml biotin labeled EGF (Life Technology, #E3477, W562-hL1-Biotin). After washing twice with cold PBS supplemented with 1% bovine serum albumin (wash buffer), cell surface bound antibody was detected by incubating the cells with Streptavidin PE (Affymetrix, #12-4317-87) for 30 minutes at 4° C. Cells were washed twice in the same buffer and the mean fluorescence (MFI) of stained cells was measured using a FACS Canto II cytometer (BD Biosciences). Wells containing no antibody or secondary antibody only were used to establish background fluorescence. Four-parameter non-linear regression analysis was used to obtain IC₅₀ values for cell binding using GraphPad Prism software.

As shown in FIG. 10b, the bispecific antibodies had similar effect as their parental antibody 305B as well as WBP305-BMK1 in blocking PD-1 binding to PDL1. The IC₅₀ of WBP336B, WBP336C, 305B and WBP305-BMK1 were 1.12, 0.79, 0.68 and 0.90 nM, respectively. The bispecific antibodies and their parental Ab could also block murine PD-1/PDL1 interaction, as shown in FIG. 10c. The IC₅₀ of WBP336B, WBP336C, 305B were 31.77, 18.73 and 16.78 nM, respectively. The antibodies blocked murine PD-1 less effective than blocking human PD-1, might due to their lower affinity to murine PD-1 than to human PD-1.

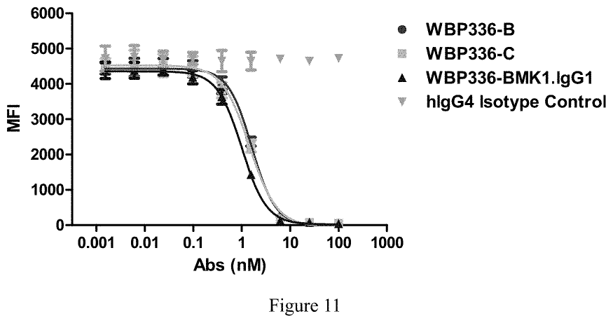

The Bispecific antibodies could also block EGF/EGFR interaction. As shown in FIG. 11, WBP336B, WBP336C and WBP336-BMK1 blocked EGF binding to EGFR at IC₅₀ of 1.62, 1.44 and 1.01 nM, respectively, indicating the bispecific antibodies maintained their potency directed against EGFR.

### 6. Cell-Based Functional Assays

Several cells based assays were conducted to evaluate the function of the Bispecific antibodies. An allogenic mixed lymphocyte reaction (MLR) assay was used to evaluate their function against PD-1. Briefly, purified CD4+ T cells were co-cultured with immature or mature allogeneic DCs (iDCs or mDCs). MLR was set up in 96-well round bottom plates using complete RPMI-1640 medium. CD4+ T cells, various concentrations of antibodies, and iDC or mDC were added to the plates. The plates were incubated at 37° C., 5% CO₂. IL-2 and IFN-γ production was determined at day 3 and day 5, respectively. The cells were harvest at day 5 to measure CD4+ T cell proliferation by ³H-TDR.

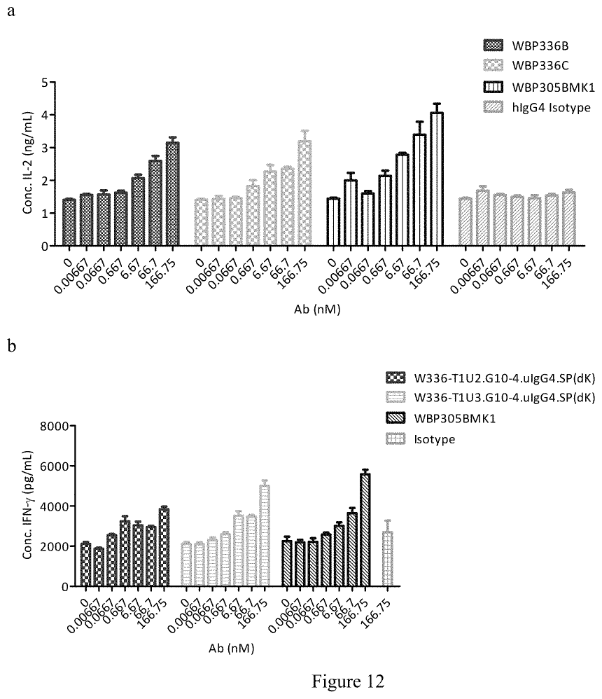

As shown in FIGS. 12a and 12b, WBP336B and WBP336C improved IL2 and INFγ release in a dose-dependent manner, similar to anti-PD-1 antibody.

The antibodies were also tested their ability to block phosphorylation of EGFR in A431 cells. Briefly, A431 cells were trypsinized, and diluted to 5×10⁵ cells/mL. A volume of 100 μL of the cell suspension was then added to each well of a 96-well clear flat bottom microplate (Corning-3599) to give a final density of 5×10⁴ cells/well. A431 cells were allowed to attach for approximately 18 hours before the media was exchanged for starvation media without fetal bovine serum. All plates were incubated overnight at 37° C. prior to treatment with the appropriate concentration of EGFR×PD-1 bispecific antibodies, EGFR monoclonal antibody or hIgG control antibody with 200 ng/ml EGF (Sino Biological-10605-HNAE) for 2 hours at 37° C. All media was gently aspirated and cells washed with ice-cold DPBS (GE-Healthcare-SH30028). The cells were lysed by adding 110 μL/well ice-cold lysis buffer (R&D System-DYC002) supplemented with 10 μg/ml Aprotinin (Thermo-Prod78432) and Leupeptin hemisulfate (Santa Cruz Biotechnology-SC-295358) and incubated on ice for 15 minutes. Store all the lysates at −80° C.

An ELISA assay was used to detect the phosphorylated EGFR. A 96-well ELISA plates (Nunc MaxiSorp, ThermoFisher) was coated overnight at room temperature with 8 g/ml human EGFR capture antibody (R&D Systems-DYC1095B). The plate was washed three times with wash buffer and blocked with 1% (w/v) bovine serum albumin (Pierce) dissolved in PBS for 1 hour at room temperature. The cell lysates were then collected and spun at 2000 μg for 5 minutes at 4° C. to remove cell debris. 100 μL supernatant were added to each well and incubated the plates for 2 hours at room temperature. Following the incubation, the plate was washed three times with 300 μL per well of PBS containing 0.5% (v/v) Tween 20. Phosphorylated EGFR was detected using anti-Phospho-tyrosine-HRP (R&D Systems-DYC1095B) by incubating at room temperature for 1 hour. The wells were washed with wash buffer three times. A volume of 100 μL per well of substrate mixture (R&D Systems-DY999) was added for the detection. The reaction was stopped after approximate 10 minutes through the addition of 50 μL per well of 2 M HCl. The absorbance of the wells was measured at 450 nm with a multi-well plate reader (SpectraMax® M5e). Four-parameter non-linear regression analysis was used to obtain IC₅₀ values for EGFR phosphorylation inhibition using GraphPad Prism software.

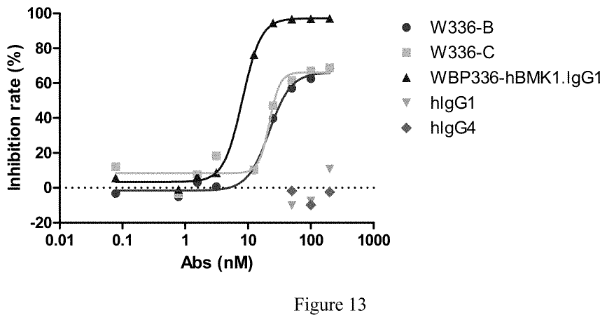

As shown in FIG. 13, the antibodies could also inhibit phosphorylation of EGFR in A431 cells in a dose dependent manner. However, the bispecific antibodies appeared less effective than their parental antibody cetuximab in inhibition of phosphorylation of EGFR, including low maximum inhibition and high IC₅₀ (21.8, 21.9 and 8.1 nM for WBP336B, WBP336C and cetuximab, respectively). This property of the bispecific antibodies may reduce skin toxicity of cetuximab [Liporini C 2013, J Pharmacol Pharmacother].

### 7. ADCC and CDC Assays on EGFR+ Cells and PD-1+ Cells

The bispecific antibody WBP336B and WBP336C were tested on mediating ADCC effect on EGFR+ A431 and HCC827 cells. Antibody dependent cell-mediated cytotoxicity and complement-dependent cytotoxicity were also tested on EGFR+ cells. Human peripheral blood mononuclear cells (PBMCs) were freshly isolated by Ficoll-Paque PLUS (GE Healthcare, #17-1440-03) density centrifugation from heparinized venous blood and then cultured overnight in complete media (RPMI1640 supplemented with 10% FBS, 100 U/ml penicillin, 100 μg/ml streptomycin). In brief, on the day of the ADCC assay, EGFR expressing target cells A431 and HCC827 (2E4/well) were plated in 110 μL with effector cells (PBMC/target cell ratio 20:1) and serial dilution of antibodies or hIgG isotype control in complete media for 4 hours at 37° C. Following incubation, the plates were centrifuged and supernatants were transferred to a clear bottom 96-well plate (Corning, #3599) and reaction mixture (Roche, #116447930, Cytotoxicity Reaction Kit) was added to each well and incubate for 15 minutes. After adding stop solution, plates were read by M5e to measure the absorbance of the samples at 492 nm and 600 nm.

Percent cytotoxicity was calculated using the equation:

% cytotoxicity=(Sample−Effector cell control−target cell control)/(Target Cell lysis−target cell control)*100%

For CDC assay, EGFR expressing target cells A431 and HCC827 (2×10⁴ cells/well) were plated in 110 μL with human normal serum (final 1:50 diluted) (Quidel, #A113) and serial dilution of antibodies or hIgG isotype control in complete media for 2 hours at 37° C. Following incubation, the plates were centrifuged and supernatants were transferred to a clear bottom 96-well plate (Corning, #3599) and reaction mixture (Roche, #116447930, Cytotoxicity Reaction Kit) was added to each well and incubate for 15 minutes. After adding stop solution, plates were read by M5e to measure the absorbance of the samples at 492 nm and 600 nm.

Percent cytotoxicity was calculated using the equation:

% cytotoxicity=(Sample−target cell control)/(Target Cell lysis−target cell control)*100%

The IC₅₀ values for killing were determined using GraphPad Prism software with values calculated using a four-parameter non-linear regression analysis.

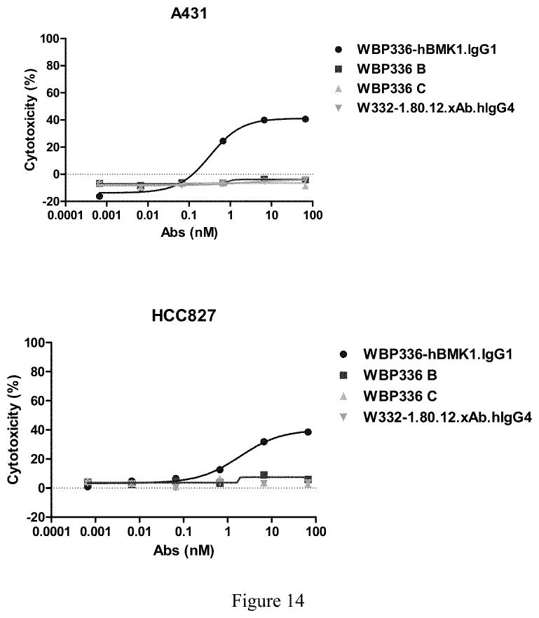

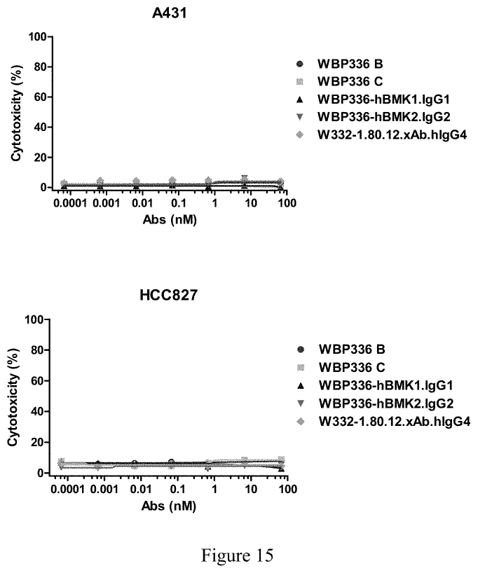

As shown in FIG. 14, the bispecific antibodies in IgG4 isotype did not induce ADCC effect on the two tumor cell lines. This property of the bispecific antibodies may reduce or avoid skin toxicity of cetuximab [Liporini C 2013, J Pharmacol Pharmacother]. The two tumor cell lines were also used to test CDC effect of the two antibodies. As shown in FIG. 15, there was no observed CDC effect of the bispecific antibodies as well as cetuximab.

Similarly, the ADCC and CDC on PD-1 positive cells were also tested. In order to test ADCC effect, activated human CD4⁺ T cells or engineered human PD-1-expressing cells WBP305.CHO-S.hPro1.C6 and various concentrations of PD-1 antibodies were pre-incubated in 96-well plate for 30 minutes, and then fresh isolated PBMCs were added at the effector/target ratio of 20:1. The plate was kept at 37° C. in a 5% CO₂ incubator for 4 hours. Target cell lysis was determined by LDH-based cytotoxicity detection kit. The absorbance was read at 492 nm using a Microplate Spectrophotometer.

For CDC, human activated CD4⁺ T cells or engineered human PD-1 expressing cells WBP305.CHO-S.hPro1.C6 and various concentrations of PD-1 antibodies were mixed in 96-well plate. Human complement was added at the dilution ratio of 1:50. The plate was kept at 37° C. in a 5% CO₂ incubator for 2 hours. Target cell lysis was determined by CellTiter-Glo.

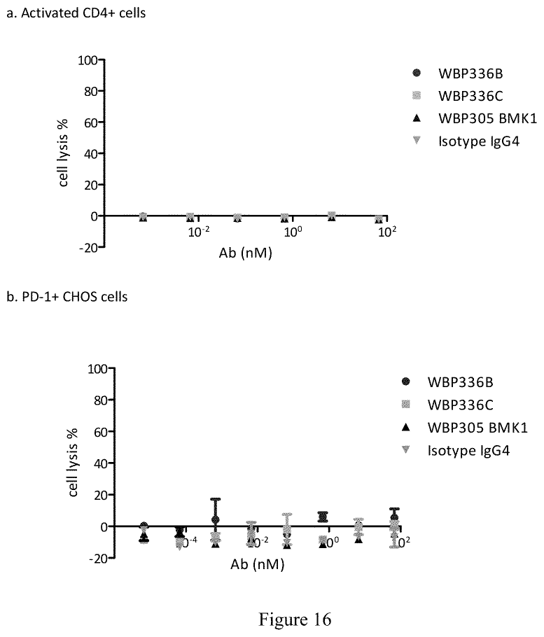

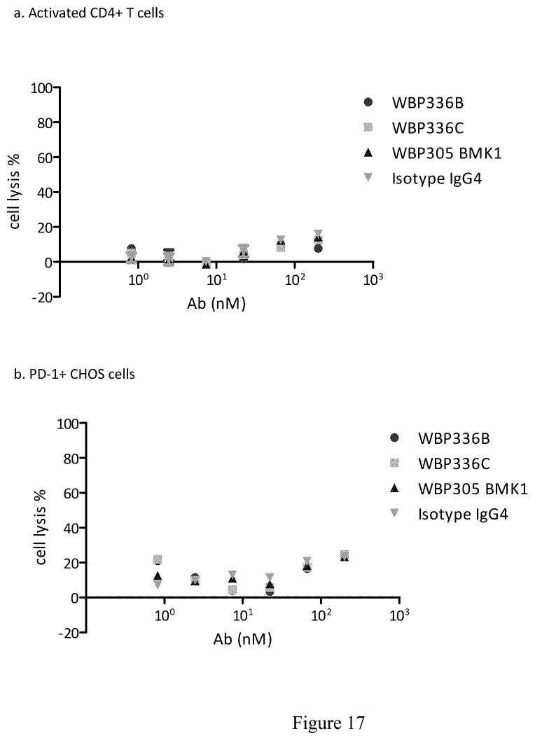

Both activated human CD4+ cells and engineered PD-1+ cells were used as target cells. As shown in FIGS. 16 and 17, the two bispecific antibodies did not induce significant ADCC or CDC effect on PD-1+ cells.

### 8. Binding to Paralogs of PD-1 and EGFR

In order to test the specificity of the two bispecific antibodies, they were tested binding on paralogs of PD-1 and EGFR. 96-well ELISA plates (Nunc MaxiSorp, ThermoFisher) were coated overnight at 4° C. with 0.5-1 μg/ml HER2-ECD or HER3-ECD in Carbonate-bicarbonate buffer. After a 1 hour blocking step with 2% (w/v) bovine serum albumin (Pierce) dissolved in PBS, serial dilutions of the different EGFR×PD-1 bispecific antibodies or positive control antibodies in PBS containing 2% bovine serum albumin were incubated on the plates for 2 hours at room temperature. Following the incubation, plates were washed three times with 300 μL per well of PBS containing 0.5% (v/v) Tween 20. Goat-anti-human IgG Fc-HRP (Bethyl, #A80-304P) at concentration of 100 ng/ml was added and incubated on the plates for 1 hour at room temperature. After washing six times with 300 L per well of PBS containing 0.5% (v/v) Tween 20, Tetramethylbenzidine (TMB) Substrate (Sigma-860336-5G) was added for the detection. The reaction was stopped after approximate 8 minutes through the addition of 100 μL per well of 2 M HCl. The absorbance of the wells was measured at 450 nm with a multiwall plate reader (SpectraMax® M5e). Non-tissue culture treated flat-bottom 96-well plates were pre-coated with 1.0 μg/ml in house made human CD28 ECD.mFc (20368), human CTLA4 ECD.his, human ICOS ECD.mFc (20374) and human PD-1 protein overnight at 4° C. After 2% BSA blocking, 100 μL 10-fold titrated antibodies from 20 nM to 0.02 pM were pipetted into each well and incubated for 1 hour at ambient temperature. Following removal of the unbound antibodies, HRP-labeled goat anti-human IgG was added to the wells and incubated for 1 hour. The color was developed by dispensing 100 μL TMB substrate, and then stopped by 100 μL 2N HCl. The absorbance was read at 450 nm using a Microplate Spectrophotometer.

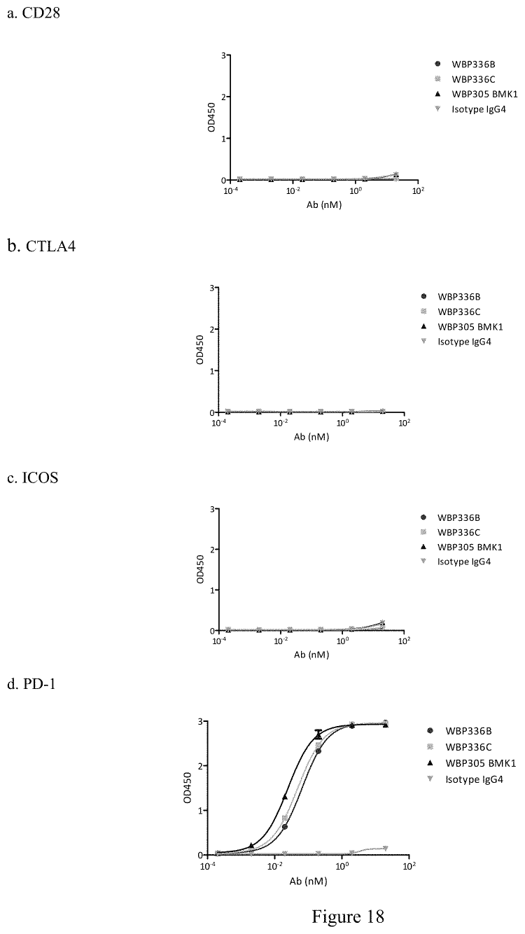

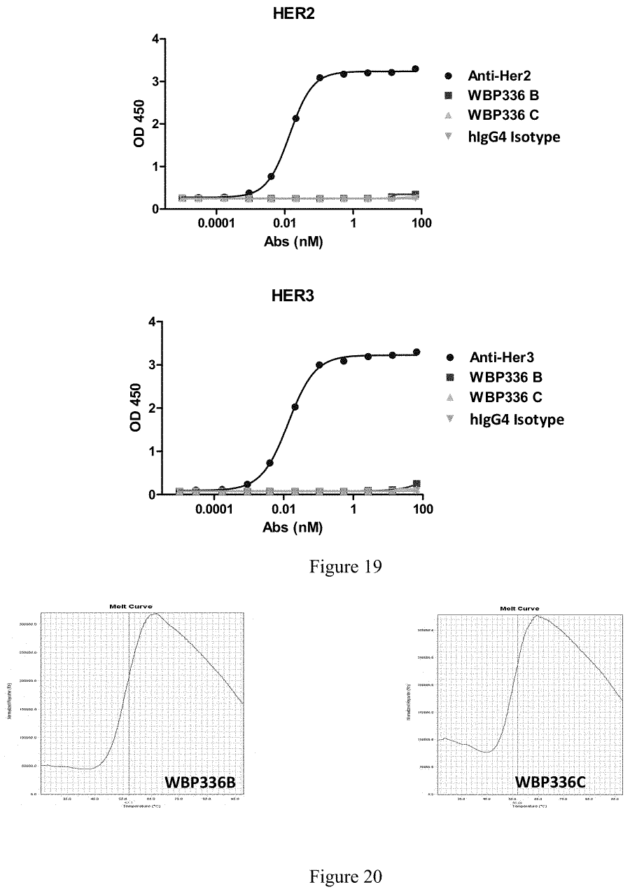

As shown in FIG. 18, the two antibodies did not bind to CD28, CTLA-4 or ICOS, the paralogs of PD-1. The antibodies did not bind to Her2 or Her3, the paralogs of EGFR (FIG. 19).

### 9. Non-Specific Binding (ELISA/FACS)

The antibodies were tested on their binding to irrelevant proteins (ELISA) or different cell lines (FACS). Both FACS and ELISA assays were used to test whether the antibodies binding to other targets. In the ELISA assay, the testing antibodies, isotype control antibodies were tested binding to different proteins including Factor VIII, FGFR-ECD, PD-1, CTLA-4.ECD, HER3.ECD, OX40.ECD, 4-1BB.ECD, CD22.ECD, CD3e.ECD, Ag1.E and XAg.ECD. Ag1.E and XAg were undisclosed proteins. Several 96-well plates (Nunc-Immuno Plate, Thermo Scientific) was coated with the individual antigens (2 μg/mL) at 4° C. overnight. After 1 hour blocking with 2% BSA in PBS, wash plate 3 times with 300 μL PBST. Testing antibodies, as well as isotype control antibodies were diluted to 10 μg/ml in PBS containing 2% BSA, then were added to the plate and incubated at room temperature for 2 hours. After 3 times washing with 300 μL PBST, HRP-conjugated goat anti-human IgG antibody (1:5000 diluted in 2% BSA) was added to the plate and incubated at room temperature for 1 hours. Finally, the plates were washed six times with 300 μL PBST. The color was developed by dispensing 100 L of TMB substrate for 12 min, and then stopped by 100 μL of 2M HCl. The absorbance at 450 nM was measured using a microplate spectrophotometer.

In FACS assay, different cell lines (Ramos, Raji, MDA-MB-453, BT474, Jurkat, Hut78, A431, A204, CaLu-6, A375, HepG2, BxPC-3, HT29, FaDu, 293F, CHO-K1) were adjusted to 1×10⁵ cells per well. Testing antibodies and Isotype control antibodies were diluted to 10 μg/ml in PBS containing 1% BSA and incubated with cells at 4° C. for 1 hr. The cells were washed twice with 180 μL PBS containing 1% BSA. PE conjugated goat anti-human IgG Fc fragment (Jackson, Catalog number 109-115-098) was diluted to final concentration 5 g/ml in PBS with 1% BSA, then added to re-suspend cells and incubated at 4° C. in the dark for 30 min. Additional washing steps were performed twice with 180 μL PBS containing 1% BSA followed by centrifugation at 1500 rpm for 4 minutes at 4° C. Finally, the cells were re-suspended in 100 μL PBS containing 1% BSA and fluorescence values were measured by flow cytometry (BD Canto II) and analyzed by FlowJo.

As shown in Table 7, among the tested proteins, WBP336B and WBP336C only bound to PD-1, as expected. They did not bind to other proteins, including CTLA-4, which is a close family member of PD-1.

In a FACS assay, WBP336B and WBP336C were tested their binding on different cell lines. As shown in Table 8, the two antibodies bound to A431, CaLu-6, BxPC-3, HT29 and FaDu, the cell lines with high level EGFR expression. They also weakly bound to BT474, A375, HepG2 and 293F, the cell lines with moderate EGFR expression. The antibodies did not bind to Ramos, Raji, MDA-MB-453, Jurkat, Hut78 and CHO-K1.

Generally, the non-specific binding test demonstrate that WBP336B and WBP336C specifically bind to EGFR and PD-1.

TABLE 7
Bispecific antibody binding to different proteins in ELISA

| | | | | | | | | | | | | No |
|---|---|---|---|---|---|---|---|---|---|---|---|---|
| Antibodies | FVIII | FGFR | Ag1.E.his | PD-1 | CTLA4 | XAg.ECD | CD22 | CD3 | HER3 | OX40 | 4-1BB | coating |
| WBP336B | 0.65 | 0.37 | 0.31 | 3.73 | 0.51 | 0.24 | 0.21 | 0.42 | 0.26 | 0.20 | 0.62 | 0.40 |
| WBP336C | 0.50 | 0.25 | 0.20 | 3.80 | 0.27 | 0.14 | 0.14 | 0.23 | 0.16 | 0.12 | 0.27 | 0.24 |
| Anti-PD-1-IgG4 | 0.39 | 0.19 | 0.09 | 3.87 | 0.13 | 0.10 | 0.11 | 0.12 | 0.10 | 0.08 | 0.10 | 0.10 |
| Human IgG4 | 0.11 | 0.19 | 0.09 | 0.11 | 0.10 | 0.08 | 0.11 | 0.11 | 0.11 | 0.09 | 0.10 | 0.11 |
| HRP-anti-hFc only | 0.08 | 0.18 | 0.06 | 0.09 | 0.08 | 0.07 | 0.09 | 0.10 | 0.08 | 0.06 | 0.08 | 0.08 |

TABLE 8
Bispecific antibody binding to different proteins in FACS (MFI value)

| | | | MDA- | | | | | | | | | | | | | |
|---|---|---|---|---|---|---|---|---|---|---|---|---|---|---|---|---|
| Antibodies | Ramos | Raji | MB-453 | BT474 | Jurkat | Hut78 | A431 | A204 | CaLu-6 | A375 | HepG2 | BxPC-3 | HT29 | FaDu | 293F | CHO-K1 |
| Blank | 30 | 29 | 33 | 28 | 22 | 29 | 25 | 24 | 25 | 37 | 32 | 34 | 25 | 23 | 31 | 32 |
| PE Anti-hIgG | 65 | 67 | 34 | 24 | 21 | 28 | 25 | 24 | 25 | 34 | 33 | 35 | 24 | 23 | 31 | 30 |
| Fc only | | | | | | | | | | | | | | | | |
| WBP336B | 102 | 228 | 138 | 475 | 40 | 150 | 9491 | 33 | 3855 | 573 | 547 | 5323 | 1290 | 4229 | 976 | 36 |
| WBP336C | 78 | 121 | 146 | 465 | 29 | 78 | 7488 | 34 | 3857 | 576 | 462 | 4505 | 1278 | 4309 | 885 | 34 |
| Anti-PD-1-IgG4 | 68 | 92 | 106 | 35 | 25 | 61 | 30 | 26 | 28 | 39 | 48 | 39 | 28 | 29 | 46 | 32 |
| Human IgG4 | 63 | 72 | 45 | 30 | 22 | 38 | 27 | 24 | 28 | 35 | 36 | 35 | 27 | 25 | 34 | 32 |

### 10. Thermal Stability

A DSF assay was used to measure the thermal stability of the bispecific antibodies. The DSF assay was performed using 7500 Fast Real-Time PCR system (Applied Biosystems). Briefly, 19 μL of bispecific antibody solution was mixed with 1 μl of 62.5×SYPRO Orange solution (TheromFisher-6650) and added to a 96 well plate. The plate was heated from 26° C. to 95° C. at a rate of 2° C./min and the resulting fluorescence data was collected. The data was analyzed automatically by its operation software and Tₕ was calculated by taking the maximal value of negative derivative of the resulting fluorescence data with respect to temperature. Tₒₙ can be roughly determined as the temperature of negative derivative plot beginning to decrease from a pre-transition baseline.

As shown in Table 9 and FIG. 20. The two antibodies have similar Th1 at 57° C.

TABLE 9
DSF data of bispecific antibodies.

| | | | | Concen- | | | |
|---|---|---|---|---|---|---|---|
| Protein | | | | tration | Tₒₙ | Tₕ 1 | Tₕ 2 |
| Name | Isotype | pI | Buffer | (mg/ml) | (° C.) | (° C.) | (° C.) |
| WBP336B | hIgG4, | 7.5 | 20 mM His, | 2.3 | 43 | 57.1 | na |
| | kappa | 0 | 5% Sucrose, | | | | |
| | | | pH 6.0 | | | | |
| WBP336C | hIgG4, | 7.5 | 20 mM His, | 2.2 | 45 | 57.0 | na |
| | kappa | 0 | 5% Sucrose, | | | | |
| | | | pH 6.0 | | | | |

### 11. Serum Stability

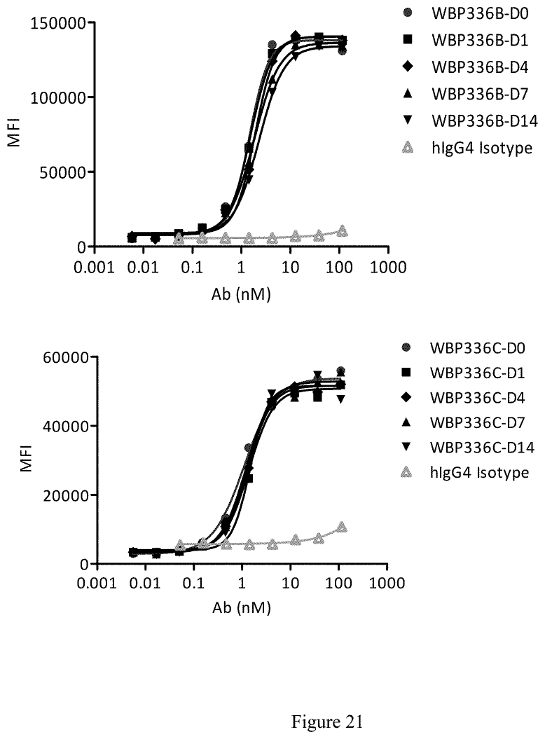

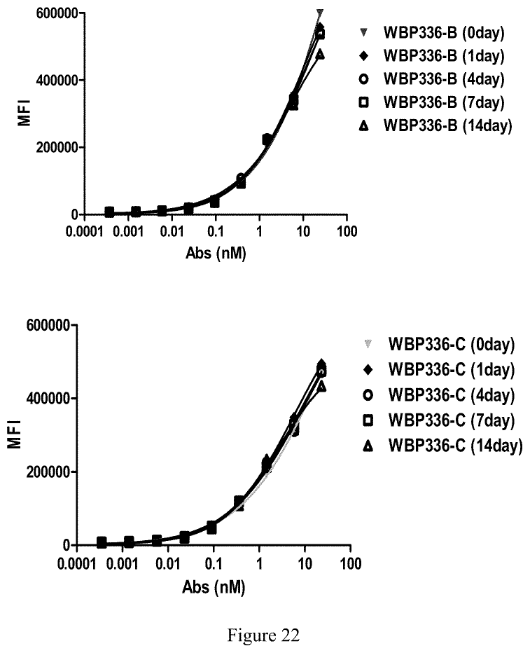

The bispecific antibodies were incubated with human serum for up to 14 days, and their binding to PD-1 and EGFR were tested from time to time. Freshly collected human blood was statically incubated in polystyrene tubes without anticoagulant for 30 minutes at room temperature. Serum was collected after centrifugation the blood at 4000 rpm for 10 minutes. The centrifugation and collection steps were repeated until the serum was clarifying. The antibodies gently mixed with serum at 37° C. for 14 days, and aliquots were drawn at the indicated time points: 0 day, 1 day, 4 days, 7 days and 14 days, and the aliquots were quickly-frozen into liquid nitrogen and store them at −80° C. until use. The samples were used to assess their binding ability on EGFR+ A431 and engineered PD-1+ CHO cells by FACS. As shown in FIGS. 21 and 22, their binding to PD-1 and EGFR did not significantly change over time, indicating that the bispecific antibodies were stable in human serum for at least 14 days.

### 12. Stress Test

WBP336B (W336-T1U2.G10-4.uIgG4.SP(dK)), WBP336 C(W336-T1U3.G10-4.uIgG4.SP(dK)), anti-EGFR antibody (WBP336-hBMK1.IgG1) and anti-PD-1 antibody were buffer exchanged into 20 mM Tris, 150 mM NaCl, pH 8.5 using Micro Float-A-Lyzer® Dialysis Device (8-10 kD, spectral/por) and further concentrated to 1 mg/ml using ultrafiltration filter (Amicon Ultra Centrifugal Filter, 30K MWCO, 0.5 mL, Merck Millipore Crop.). Quantification of antibody was performed using Uv-Vis spectrophotometer (NanoDrop 2000 Spectrophotometer, Thermo Scientific Inc.). Antibody was incubated at 37° C. and withdrawn after 5 days of incubation for analysis of target-binding by surface plasmon resonance (SPR). The interaction between the antibodies and two antigens (PD1.ECD.his and EGFR.ECD.his) was measured by SPR (ProteOn XPR36, Bio-Rad Laboratories, Inc.). Each antibody was captured onto the anti-human Fc IgG (Jackson, Cat. No.: 109-005-098) surface immobilized on GLM-biosensor chip (Bio-Rad Laboratories, Inc.). The assay was performed at 25° C. with 1×PBST as running and dilution buffer. 1:5 serially diluted W305-hPro1.ECD.his solutions (20, 10, 5, 2.5 and 1.25 nM) and running buffer were injected at a flow rate of 100 μL/min for an association phase of 120 s, followed by 400 s dissociation. Regeneration of the chip surface was reached by an 18-s injection of 10 mM Glycine, pH 1.5. After regeneration, 1:5 serially diluted W562-hPro1.ECD.his solutions (20, 10, 5, 2.5 and 1.25 nM) and running buffer were injected at a flow rate of 100 μL/min for an association phase of 120 s, followed by 1200 s dissociation. Regeneration of the chip surface was reached by an 18 s-injection of 10 mM Glycine, pH 1.5.

As there are potential PTM sites on WBP336B and WBP336C (Table 3), these antibodies were tested their resistance to high pH and high temperature conditions. These antibodies were incubated at pH 8.0 and 37° C. for 5 days, and their binding on PD-1 and EGFR were measured using SPR.

As shown in Table 10 and 11, their binding on PD-1 or EGFR did not change in the high pH and high temperature conditions, indicating there were no significant PTM or the PTM did not change their binding activity to the targets.

TABLE 10
Antibody binding on PD-1

| Ligand | conditions | Ka (1/Ms) | kd (1/s) | K_{D} (M) |
|---|---|---|---|---|
| WBP336B | 37° C., pH8 | 1.20E+06 | 8.31E−03 | 6.92E−09 |
| | 4° C., pH 6.5 | 1.27E+06 | 9.75E−03 | 7.68E−09 |
| WBP336C | 37° C., pH8 | 1.15E+06 | 2.06E−03 | 1.79E−09 |
| | 4° C., pH 6.5 | 1.21E+06 | 2.40E−03 | 1.98E−09 |
| Anti-PD-1 | 37° C., pH8 | 8.42E+05 | 3.62E−04 | 4.30E−10 |
| | 4° C., pH 6.5 | 8.03E+05 | 5.19E−04 | 6.47E−10 |
| cetuximab | 37° C., pH8 | no binding | | |
| | 4° C., pH 6.5 | no binding | | |

TABLE 11
Antibody-binding on EGFR

| Ligand | conditions | ka (1/Ms) | kd (1/s) | K_{D} (M) |
|---|---|---|---|---|
| WBP336B | 37° C.. pH8 | 1.35E+06 | 7.27E−04 | 5.40E−10 |
| | 4° C., pH 6.5 | 1.30E+06 | 7.16E−04 | 5.49E−10 |
| WBP336C | 37° C., pH8 | 1.35E+06 | 7.46E−04 | 5.54E−10 |
| | 4° C., pH 6.5 | 1.36E+06 | 7.41E−04 | 5.45E−10 |
| Cetuximab | 37° C., pH8 | 1.23E+06 | 6.68E−04 | 5.44E−10 |
| | 4° C., pH 6.5 | 1.19E+06 | 6.46E−04 | 5.45E−10 |
| Anti-PD-1 | 37° C., pH8 | no binding | | |
| | 4° C., pH 6.5 | no binding | | |

### 13. Granzyme B Assay

EGFR expressing A431 cells (5×10³ cells/well in 50 μL) were plated with PBMCs or CD8+ T cells (1×10⁵ cells/well in 50 μL, activated by 25 ng/mL PMA and 50 ng/mL Ionomycin) for 7 days and then with antibodies or hIgG Isotype control in 100 μL complete media for 24 hours at 37□. Following incubation, the plates were centrifuged and supernatants were transferred to clear bottom 96-well plates (Corning, #3799). The cells were resuspended in 100 μL R&D lysis buffer (Cat: DYC002) with 10 μg/mL Aprotinin and 10 μg/mL Leupeptin and put on ice for 20 mins. Before detecting Granzyme B, the samples were centrifuged at approximately 10000 g for 5 min and the cell lysates were collected. Two-fold titrated standard from 8000 pg/mL to 15.36 pg/mL, diluted supernatant and diluted cell lysates were added 100 μL per well into ELISA plates. After incubation at 37° C. for 1.5 hours, biotinylated anti-Human Granzyme B antibody was added 100 μL per well and incubated at 37° C. for 1 hour. The plates were washed 3 times and prepared 100 μL Avidin-Biotin-Peroxidase Complex working solution were added into each well. Another 5 times of washing step were performed following 30 min incubation at 37° C. The absorbance at 450 nm was measured using a microplate reader within 30 min after stop the TMB color developing.

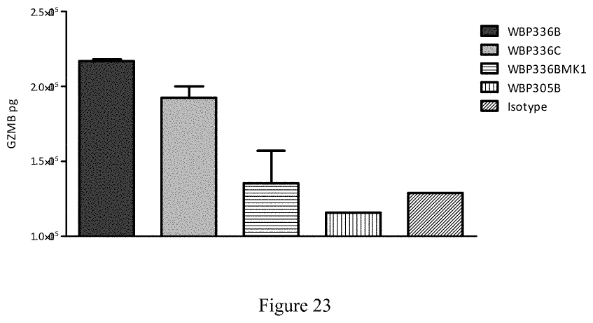

The results of bispecific antibody WBP336B/WBP336C increased Granzyme B secretion were shown in FIG. 23. Compared with anti-EGFR antibody, anti-PD-1 antibody and isotype control, the bispecific antibodies WBP336B or WBP336C could promote Granzyme B secretion.

### Example 5: In Vivo Characterization

### 1. Efficacy in A431 Xenograft Mouse Model

The A431 tumor cells (ATCC, Manassas, Va., cat #CRL-1555) were maintained in vitro as a monolayer culture in 1640 medium supplemented with 15% heat inactivated fetal calf serum, 100 U/mL penicillin and 100 μg/ml streptomycin at 37° C. in an atmosphere of 5% CO₂ in air. The tumor cells were routinely subcultured twice weekly. The cells growing in an exponential growth phase were harvested and counted for tumor inoculation.

PBMCs were collected from whole blood donated by healthy donor and extracted using 1.077 Ficoll (GE Healthcare company, GE Healthcare), a hydrophilic polysaccharide that separates layers of blood. A gradient centrifugation separated the blood into a top layer of plasma, followed by a layer of PBMCs and a bottom fraction of polymorphonuclear cells and erythrocytes. Freshly isolated PBMCs were co-cultured with mytomycin treated A431 for 72 hours before inoculation, then mixed with untreated A431 with E:T ratio of 1:3.

Each mouse was inoculated subcutaneously at the right flank with A431 tumor cells (5×10⁶) co-cultured 3-4 days with or without PBMC (1.67×10⁶) in 0.2 mL of PBS for tumor development. The treatments were started on day 3 after tumor inoculation when the average tumor size reached approximately 60 mm³. The mice number of each group and testing article were administrated to the mice according to the predetermined regimen.

All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC). At the time of routine monitoring, the animals were daily checked for any effects of tumor growth and treatments on normal behavior such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset.

The major endpoint was to see if the tumor growth could be delayed or mice could be cured. Tumor size was measured twice weekly in two dimensions using a caliper, and the volume was expressed in mm³ using the formula: V=0.5 a×b² where a and b are the long and short diameters of the tumor, respectively. The T/C value (in percent) is an indication of antitumor effectiveness.

TGI was calculated for each group using the formula: TGI (%)=[1−(Ti−T0)/(Vi-V0)]×100, whereas Ti is the average tumor volume of a treatment group on a given day, T0 is the average tumor volume of the treatment group on the day of treatment start, Vi is the average tumor volume of the vehicle control group on the same day with Ti, and V0 is the average tumor volume of the vehicle group on the day of treatment start.

### Statistical Analysis

Summary statistics, including mean and the standard error of the mean (SEM), are provided for the tumor volume of each group at each time point. Statistical analysis of difference in tumor volume among the groups and the analysis of drug interaction were conducted on the data obtained at the best therapeutic time point after the final dose (the 28^{th} day after start dosing).

A one-way ANOVA was performed to compare tumor volume among groups, followed by post-hoc multiple comparison of Dunnett't test (all compared to IgG group). All data were analyzed using SPSS 17.0. p<0.05 was considered to be statistically significant.

TABLE 12
Tumor growth inhibition

| | Tumor | | | |
|---|---|---|---|---|
| | Size | | | |
| | (mm³)^{a} | T/C^{b} | TGI^{b} | p value^{c} |
| Treatment | at day 28 | (%) | (%) | (vs. hIgG4) |
| Control hIgG4 | 2191 ± 869 | — | — | — |
| Anti-PD-1 (WBP305B) | 1389 ± 317 | 103.00 | 37.55 | 0.163 |
| Anti-EGFR | 0 ± 0 | 0.00 | 102.67 | 0.000 |
| WBP336B, 5 mpk | 18 ± 10 | 1.37 | 101.78 | 0.000 |
| WBP336B, 10 mpk | 0 ± 0 | 0.00 | 102.69 | 0.000 |

Note:
^{a}Mean ± SEM.
^{b}Tumor Growth Inhibition is calculated by dividing the group average tumor volume for the treated group by the group average tumor volume for the control group (T/C and TGI). For a test article to be considered to have anti-tumor activity, T/C must be 50% or less.
^{c}p value is calculated based on tumor size.

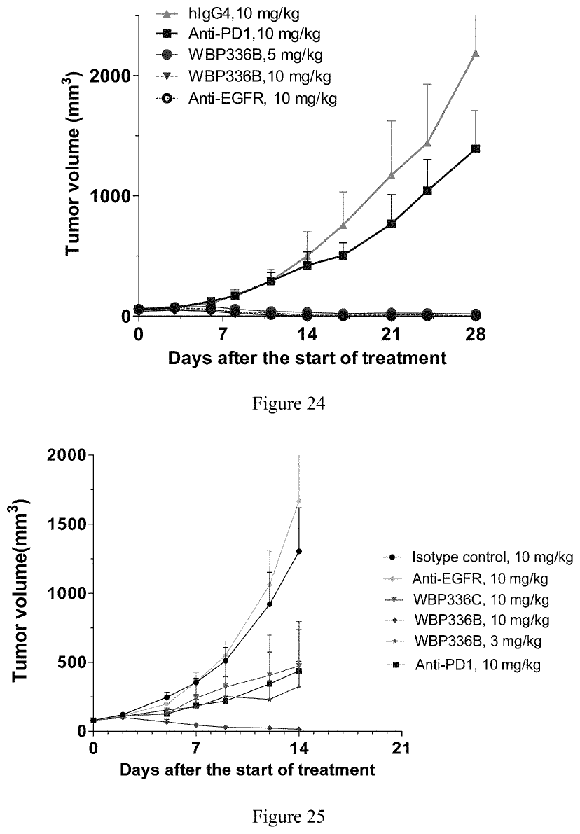

WBP336B or control antibodies was injected twice weekly into the mice of different groups. Tumor were measured three times a week, and the results are shown in FIG. 24. Tumor growth inhibition in MiXeno humanized mice bearing A431 xenografts was calculated based on tumor volume measurements at day 28. Compared with isotype control (hIgG4) group, anti-PD-1 antibody group slightly inhibited tumor growth (p=0.163). In contrast, anti-EGFR (cetuximab) group as well as WBP336B group completely induced tumor regression (p=0.000), as analyzed in Table 12. The results indicate that the anti-EGFR activity of WBP336B completely inhibited tumor growth.

### 2. Efficacy in MC38 Synergetic Mouse Model

To test anti-PD-1 activity of the bispecific antibodies in vivo, we used a syngeneic mouse model due to the bispecific antibodies' cross-reactivity to murine PD-1.

The MC38 cell was maintained in vitro as a monolayer culture in DMEM medium supplemented with 10% fetal bovine serum, 2 mM L-glutamine, 100 U/ml penicillin and 100 g/mL streptomycin at 37° C. in an atmosphere of 5% CO₂ in air. The tumor cell was routinely subcultured twice weekly by trypsin-EDTA treatment. The cell growing in an exponential growth phase was harvested and counted for tumor inoculation.

Each mouse was inoculated subcutaneously at the right axillary (lateral) with MC38 tumor cell (3×10⁵) in 0.1 mL of PBS for tumor development. The animals were randomly grouped when the average tumor volume reached 79 mm³, then treatment started for the efficacy study.

All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC). At the time of routine monitoring, the animals were daily checked for any effects of tumor growth and treatments on normal behavior such as mobility, food and water consumption (by looking only), body weight gain/loss (body weights were measured once every day), eye/hair matting and any other abnormal effect as stated in the protocol. Death and observed clinical signs were recorded on the basis of the numbers of animals within each subset.

The major endpoint was to see if the tumor growth could be delayed or mice could be cured. Tumor sizes were measured three times weekly in two dimensions using a caliper, and the volume was expressed in mm³ using the formula: V=0.5 a×b² where a and b were the long and short diameters of the tumor, respectively. The tumor sizes were then used for the calculations of T/C (%) values. The T/C value (in percent) is an indication of antitumor effectiveness, T and C are the mean volume of the treated and control groups, respectively, on a given day.

TGI was calculated for each group using the formula: TGI (%)=[1−(Ti−T0)/(Vi−V0)]×100; Ti is the average tumor volume of a treatment group on a given day, T0 is the average tumor volume of the treatment group on the first day of treatment, Vi is the average tumor volume of the vehicle control group on the same day with Ti, and V0 is the average tumor volume of the vehicle group on the first day of treatment.

Summary statistics, including mean and the standard error of the mean (SEM), were provided for the tumor volume of each group at each time point. Statistical analysis of difference in tumor volume among the groups and the analysis of drug interaction were conducted on the data obtained at the best therapeutic time point on the 14th day after the start of treatment.

One-way ANOVA was performed to compare tumor volume among groups, and when a significant F-statistics (a ratio of treatment variance to the error variance) was obtained, comparisons between groups were carried out with Games-Howell test. For comparison between two groups, Mann-Whitney test was used. All data were analyzed using SPSS 17.0 and prism 5. p<0.05 was considered to be statistically significant.

The results are shown in FIG. 25. Both WBP336B and WBP336C significantly inhibit tumor growth (p<0.05), and WBP336B induced tumor regression. As the anti-human EGFR antibody does not bind to murine EGFR, the anti-human EGFR antibody did not show any anti-tumor effect in this model, which demonstrated that the anti-tumor effect of the bispecific antibody mainly contributed from anti-PD-1 arm.

### 3. Antibody Bio-Distribution in A431/hPBMC Mouse Model

### 3.1 Cell Culture

The A431 tumor cells (ATCC, cat #CRL-1555) were maintained in vitro as a monolayer culture in 1640 medium supplemented with 15% heat inactivated fetal calf serum, 100 U/mL penicillin and 100 μg/mL streptomycin at 37° C. in an atmosphere of 5% CO₂ in air. The tumor cells were routinely subcultured twice weekly. The cells growing in an exponential growth phase were harvested and counted for tumor inoculation.

### 3.2 Peripheral Blood Mononuclear Cell (PBMC) Extraction

PBMCs were collected from whole blood donated by healthy donor and extracted using 1.077 Ficoll (GE Healthcare company, GE Healthcare), a hydrophilic polysaccharide that separates layers of blood. A gradient centrifugation separated the blood into a top layer of plasma, followed by a layer of PBMCs and a bottom fraction of polymorphonuclear cells and erythrocytes.

### 3.3 Tumor and PBMC Inoculation for MiXeno Subcutaneous Xenegraft Model

Each mouse was inoculated subcutaneously at the right flank with A431 tumor cells (5×10⁶) at day 0. When the average tumor size reached approximately 50 mm³, PBMC (3×10⁶) in 0.2 mL of PBS iv. Injected into each mice. The treatments were started when the average tumor size reached approximately 600 mm³. The mice number of each group and testing article were administrated to the mice according to the predetermined regimen as shown in the experimental design table below.

### 3.4 Experimental Design Table

TABLE 13
Experimental design

| | | | Dose | Dosing | Dosing |
|---|---|---|---|---|---|
| Group | n | Treatment | (mg/kg) | route | Schedule |
| 1 | 3 | Isotype | 10 | I.V. | Single dose |
| 2 | 3 | Anti-PD-1 | 10 | I.V. | Single dose |
| 3 | 3 | Anti-EGFR | 10 | I.V. | Single dose |
| 4 | 3 | WBP336B | 10 | I.V. | Single dose |
| 5 | 3 | WBP336C | 10 | I.V. | Single dose |

### 3.5 Observations

All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC). At the time of routine monitoring, the animals were daily checked for any effects of tumor growth and treatments on normal behavior such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset.

### 3.6 Samples Collection and Preparation at Different Time Points

After antibody injection, blood and tissue samples were collected at 48 h, 72 hand 6 days' time point. Tumor and liver samples were collected to test antibody by HC. Before tissue samples collection, PBS perfusion was used to get rid of blood from tissues. Approximately 60-90 mg tumor and liver samples were embedded in OCT for IHC staining.

### 3.7 Results

As shown in table 14, the isotype control, anti-PD-1 antibody had similar IHC score in liver and tumor tissue. Whereas the anti-EGFR antibody and bispecific antibody WBP336B/C had higher IHC score in tumor than in liver tissue. The results indicate that the bispecific antibodies preferential distribute in tumor tissue.

TABLE 14

| | Mouse | Score for | Score for |
|---|---|---|---|
| Group ID | ID | liver | tumor |
| Isotype | #347 | 1+ | 1+ |
| | #356 | 2+ | 1+ |
| | #358 | 1+ | 1+ |
| Anti-PD-1 | #341 | 1+ | 2+ |
| | #346 | 1+ | 1+ |
| | #359 | 1+ | 1+ |
| anti-EGFR | #344 | 1+ | 3+ |
| | #345 | 1+ | 3+ |
| | #351 | 1+ | 3+ |
| WBP336B | #342 | 1+ | 2+ |
| | #350 | 0 | 1+ |
| | #360 | 0 | 2+ |
| WBP336C | #343 | 1+ | 3+ |
| | #348 | 2+ | 3+ |
| | #352 | 1+ | 3+ |

### SEQUENCE LISTING

The sequence listing submitted herewith in the ASCII text file entitled “127501-003US1 Sequence Listing,” created Jul. 13, 2020, with a file size of 31.811 kilobytes, is incorporated herein by reference in its entirety.

1. A bispecific antibody or an antigen binding fragment thereof, comprising:
a first binding domain which binds to human EGFR, and
a second binding domain which binds to human or murine PD-1 comprising the single chain Fv against PD-1;
wherein the single chain Fv against PD-1 comprises a VH region and a VL region against PD-1, the VH region against PD-1 comprising H-CDR1, H-CDR2, H-CDR3 and a VL region against PD-1 comprising L-CDR1, L-CDR2, L-CDR3;
wherein the H-CDR3 comprises an amino acid sequence as depicted in SEQ ID NO: 14 or SEQ ID NO: 18, and conservative modifications thereof; the H-CDR2 comprises an amino acid sequence as depicted in SEQ ID NO: 13, and conservative modifications thereof the H-CDR1 comprises an amino acid sequence as depicted in SEQ ID NO: 12, and conservative modifications thereof;
the L-CDR3 comprises an amino acid sequence as depicted in SEQ ID NO: 17, and conservative modifications thereof; L-CDR2 comprises an amino acid sequence as depicted in SEQ ID NO: 16, and conservative modifications thereof; the L-CDR1 comprises an amino acid sequence as depicted in SEQ ID NO: 15, and conservative modifications thereof.

2. The antibody or the antigen binding-fragment thereof according to claim 1,
wherein the antibody or the antigen binding-fragment comprises a format selected from the group consisting of
i) a first polypeptide chain comprising, from N-terminus to C-terminus,
a single chain Fv against human EGFR, operably linked to an antibody light chain constant (CL) domain; and
second polypeptide chain comprising, from N-terminus to C-terminus, a single chain Fv against human or murine PD-1, operably linked to an antibody heavy chain constant (CH) domain;
(ii) a first polypeptide chain comprising, from N-terminus to C-terminus, a VL region against EGFR, operably linked to an antibody light chain constant (CL) domain and the single chain Fv against PD-1; and
a second polypeptide chain comprising, from N-terminus to C-terminus, a VH region against EGFR, operably linked to an antibody heavy chain constant (CH) domain;
(iii) a first polypeptide chain comprising, from N-terminus to C-terminus, a VL region against EGFR, operably linked to an antibody light chain constant (CL) domain; and
a second polypeptide chain comprising, from N-terminus to C-terminus, a VH region against EGFR, operably linked to an antibody heavy chain constant (CH) domain and the single chain Fv against PD-1.

3. The antibody or the antigen binding fragment thereof according to claim 1
wherein the antibody or the antigen binding-fragment comprises a format:
a first polypeptide chain comprising, from N-terminus to C-terminus, a single chain Fv against human EGFR, operably linked to an antibody light chain constant (CL) domain; and
a second polypeptide chain comprising, from N-terminus to C-terminus, a single chain Fv against human or murine PD-1, operably linked to an antibody heavy chain constant (CH) domain.

4. The antibody or the antigen binding fragment thereof according to claim 1 wherein the single chain Fv against PD-1 comprises:
(i) an amino acid sequence that is at least 70%, 80%, 90%, 95% or 99% homologous to a sequence selected from a group consisting of SEQ ID NOs: 1, 3; or
(ii) an amino acid sequence selected from the group consisting of SEQ ID NOs: 1, 3.

5. The antibody or the antigen binding fragment thereof according to claim 3 wherein the second polypeptide chain comprises:
(i) an amino acid sequence that is at least 70%, 80%, 90%, 95% or 99% homologous to a sequence selected from a group consisting of SEQ ID NOs: 19, 21; or
(ii) an amino acid sequence selected from the group consisting of SEQ ID NOs: 19, 21.

6. The antibody or an antigen binding fragment thereof according to claim 1, wherein the first binding domain which binds to human EGFR comprises
a VH region comprising H-CDR1, H-CDR2, H-CDR3 and a VL region comprising L-CDR1, L-CDR2, L-CDR3; wherein
the H-CDR3 comprises a sequence as depicted in SEQ ID NO: 8, and conservative modifications thereof; the H-CDR2 comprises a sequence as depicted in SEQ ID NO: 7, and conservative modifications thereof; the H-CDR1 comprises a sequence as depicted in SEQ ID NO: 6, and conservative modifications thereof; and
the L-CDR3 comprises a sequence as depicted in SEQ ID NO: 11, and conservative modifications thereof; the L-CDR2 comprises a sequence as depicted in SEQ ID NO: 10, and conservative modifications thereof; the L-CDR1 comprises a sequence as depicted in SEQ ID NO: 9, and conservative modifications thereof.

7. The antibody or an antigen binding fragment thereof according to claim 3 wherein the single chain Fv against human EGFR comprises:
(i) an amino acid sequence that is at least 70%, 80%, 90%, 95% or 99% homologous to a sequence selected from a group consisting of SEQ ID NOs: 2, 4, 5;
(ii) an amino acid sequence selected from the group consisting of SEQ ID NOs:2, 4, 5.

8. The antibody or the antigen binding-fragment thereof according to claim 7 wherein the first polypeptide chain comprises
(i) an amino acid sequence that is at least 70%, 80%, 90%, 95% or 99% homologous to a sequence selected from a group consisting of SEQ ID NOs: 20, 22, 23; or
(ii) an amino acid sequence selected from the group consisting of SEQ ID NOs: 20, 22, 23.

9. The antibody or the antigen binding-fragment thereof according to claim 3, wherein
the first polypeptide chain comprises: an amino acid sequence selected from the group consisting of SEQ ID NOs: 20, 22, 23.
the second polypeptide chain comprises: an amino acid sequence selected from the group consisting of SEQ ID NOs: 19, 21.

10. The antibody or the antigen binding-fragment thereof according to claim 1, wherein the antibody is chimeric, humanized, fully human, or rodent antibody.

11. A nucleic acid molecule encoding antibody or the antigen binding-fragment thereof according to claim 1.

12. A cloning or expression vector, comprising the nucleic acid molecule of claim 11.

13. A host cell comprising one or more cloning or expression vectors of claim 12.

14. A process for the production of the antibody of claim 1, comprising culturing the host cell comprising a nucleic acid molecule encoding the antibody or the antigen binding fragment thereof, and isolating the antibody.

15. A pharmaceutical composition comprising the antibody or the antigen binding-fragment thereof according to claim 1, and one or more of a pharmaceutically acceptable excipient, a diluent and a carrier.

16. An immunoconjugate comprising the antibody or the antigen binding fragment thereof according to claim 1, linked to a therapeutic agent.

17. A method of modulating an immune response in a subject comprising administering to the subject the antibody or the antigen binding fragment thereof according to claim 1.

18. A method of inhibiting growth of tumor cells in a subject, comprising administering to the subject a therapeutically effective amount of the antibody or the antigen binding fragment thereof according to claim 1, to inhibit growth of the tumor cells.

19-31. (canceled)

32. The method of claim 18, wherein the tumor cells are of a cancer selected from a group consisting of melanoma, renal cancer, prostate cancer, breast cancer, colon cancer, lung cancer, bone cancer, pancreatic cancer, skin cancer, cancer of the head or neck, cutaneous or intraocular malignant melanoma, uterine cancer, ovarian cancer, and rectal cancer.

33. A kit comprising the antibody or the antigen binding fragment thereof of claim 1 and instructions for using the antibody or the antigen binding fragment thereof for detection, diagnosis, prognosis, or treatment of a EGFR-related disease or condition.
